(19) United States
(12) Patent Application Publication
(10) Pub. No.: US 2022/0339294 A1
(43) Pub. Date: Oct. 27, 2022

(54) NANOPARTICLES FOR SELECTIVE TISSUE OR CELLULAR UPTAKE

(71) Applicant: Yale University, New Haven, CT (US)

(72) Inventors: Hanna Mandl, New Haven, CT (US); Elias Quijano, New Haven, CT (US); W. Mark Saltzman, New Haven, CT (US); Peter Glazer, New Haven, CT (US)

(21) Appl. No.: 17/641,516

(22) PCT Filed: Sep. 9, 2020
(86) PCT No.: PCT/US2020/049969

§ 371 Date: Mar. 9, 2022

Related U.S. Application Data
(60) Provisional application No. 62/897,655, filed on Sep. 9, 2019.

(51) Int. Cl. A61K 47/69; A61K 9/00; A61K 9/51

(52) U.S. Cl. CPC A61K 47/6937; A61K 9/0019; A61K 9/5153; B82Y 5/00

(57) ABSTRACT

Compositions containing populations of nanoparticles that show selective uptake by tissues and other cell types such as lung cells and/or bone marrow cells are described. The nanoparticles show this uptake by virtue of their size and in the absence of a targeting agent on the surface of the nanoparticles, i.e., passive targeting. The population of nanoparticles contain poly(lactic acid-co-glycolic acid), have a diameter between about 70 nm and about 220 nm, and at least 90% of the nanoparticles have a diameter between about 110 nm and about 129 nm. The nanoparticles are manufactured using a microfluidic system. The compositions can be used to treat lung- and/or blood-related genetic disorders in in vivo gene editing technologies.

### CROSS-REFERENCE TO RELATED APPLICATION

The present application claims priority to U.S. Application No. 62/897,655, filed Sep. 9, 2019, the disclosure of which is incorporated herein by reference in its entirety.

### STATEMENT REGARDING FEDERALLY SPONSORED RESEARCH

This invention was made with government support under Grant No. T32GM07205 awarded by the NIGMS Medical Scientist Training Program, Grant No. 5T32GM007223-43, an institutional training grant, awarded by the Institute of General Medical Sciences, and Grant No. UG3 HL147352 awarded by the National Institutes of Health. The government has certain rights in the invention.

### REFERENCE TO THE SEQUENCE LISTING

The Sequence Listing submitted as a text file named “YU_7724_PCT_ST25” created on Sep. 9, 2020, and having a size of 39,693 bytes is hereby incorporated by reference pursuant to 37 C.F.R. § 1.52(e)(5).

### FIELD OF THE INVENTION

This invention is generally related to compositions for selective tissue or cellular uptake, particularly nanoparticles containing therapeutic, diagnostic, and/or prophylactic agents, selectively taken up by lung cells and/or bone marrow cells, and methods of use thereof.

### BACKGROUND OF THE INVENTION

Nanoparticles (NPs) hold great potential for controlled spatial and temporal delivery of a wide variety of therapeutic agents to disease-afflicted organs in vivo (Salata, Journal of Nanobiotechnology 2004, 2(1): 3; Lee and Yeo, Chemical Engineering Science 2015, 125, 75-84). Localizing agents to therapeutic target sites can circumvent toxicity, unwanted side effects, unnecessarily high systemic doses, and widespread distribution of precious therapeutic cargo. However, achieving successful and tissue-specific delivery is difficult. NPs can be rapidly cleared from the bloodstream by the mononuclear phagocyte system (MPS), renal filtration, and endogenous enzymes (Albanese, et al., Annu. Rev. Biomed. Eng. 2012, 14(1): 1-16; Owens and Peppas, International Journal of Pharmaceutics 2006, 307(1): 93-102). To overcome these processes and avoid non-specific uptake, NPs must be strategically engineered to reach their site of therapeutic action.

Altering NP size has been shown to control tissue-specific accumulation (Lundy, et al., Scientific Reports 2016, 6: 25613; Hoshyar, et al., Nanomedicine (London, England) 2016, 11(6): 673-692; Phillips, et al., Nano Today 2010, 5(2): 143-159). However, the effectiveness of MPS uptake, renal filtration, and liver and spleen sequestration restricts the range of NP diameters that are able to access target-sites in substantial and impactful quantities. NPs with a hydrodynamic diameter greater than 200 nm, for example, are rapidly cleared from circulation and accumulate in liver and spleen tissue (Albanese, et al., Annu. Rev. Biomed. Eng. 2012, 14(1): 1-16; Owens and Peppas, International Journal of Pharmaceutics 2006, 307(1): 93-102; Hoshyar, et al., Nanomedicine (London, England) 2016, 11(6): 673-692; Bertrand and Leroux, J Control Release 2012, 161(2): 15263). This clearance is further promoted by the phagocytic action of Kupffer cells in the liver, which account for 80-90% of the total body macrophage population (Bertrand and Leroux, J Control Release 2012, 161(2): 15263). Though critical to the body's defense, these clearance processes limit the variety of NP sizes that can successfully reach their target sites in vivo.

While prior studies have evaluated the effect of NP size on in vivo biodistribution following intravenous administration (De Jong, et al., Biomaterials 2008, 29(12): 1912-1919; Pérez-Campaña, et al., ACS Nano 2013, 7(4): 3498-3505; Liao, et al., Nanoscale 2013, 5(22): 11079-11086), most have focused on inorganic NPs (De Jong, et al., Biomaterials 2008, 29(12): 1912-1919; Huang, et al., ACS Nano 2010, 4(12): 7151-7160). Although they are readily available from a variety of commercial sources, and in a variety of sizes, these NPs are not easily used to encapsulate and deliver therapeutic cargo. Contrastingly, it has previously been shown that biodegradable NPs made of poly(lactic-co-glycolic acid) (PLGA) can efficiently and safely deliver a variety of biological agents including chemotherapy drugs (Sawyer, et al., Drug Delivery and Translational Research 2011, 1(1): 34-42; Malinovskaya, et al., International Journal of Pharmaceutics 2017, 524(1): 77-90; Bowerman, et al., Nano Letters 2017, 17(1): 242-248; Householder, et al., International Journal of Pharmaceutics 2015, 479(2): 374-380), plasmid DNA (pDNA) (Blum and Saltzman, Journal of Controlled Release 2008, 129(1): 66-72; Zhao, et al., PLOS ONE 2013. 8(12): e82648; Santos, et al., Nanotechnology, Biology and Medicine 2013, 9(7): 985-995), small-interfering RNAs (siRNAs) (Woodrow, et al., Nature Materials 2009, 8: 526; Cun, et al., International Journal of Pharmaceutics 2010, 390(1): 70-75), and peptide nucleic acids (PNAs) along with donor DNA molecules for genome modification (McNeer, et al., Nature communications 2015, 6: 6952-6952; McNeer, et al., Molecular Therapy 2011, 19(1): 172-180; McNeer, et al., Gene Therapy 2012, 20: 658; Schleifman, et al., Molecular therapy. Nucleic Acids 2013, 2(11): e135-e135; Fields, et al., Advanced Healthcare Materials 2015, 4(3): 361-366; Bahal, et al., Nature Communications 2016, 7: 13304; Ricciardi, et al., Nature Communications 2018, 9(1): 2481). Yet, the exact PLGA NP size that provides effective delivery of these biological agents in vivo is not understood.

Control of NP size requires careful selection of the formulation method and control of relevant parameters. The most widely used methods to formulate NPs for biodistribution studies include double emulsion, nanoprecipitation, high-pressure homogenization, and spray drying (Huang and Zhang, Biotechnol J 2018, 13(1); Operti, et al., International Journal of Pharmaceutics 2018, 550(1), 140-148; Dong, et al., International Journal of Pharmaceutics 2007, 342(1), 208-214). Though effective, it is difficult to generate NPs with a control of size over a wide range with these approaches (Huang and Zhang, Biotechnol J 2018, 13(1)). Double emulsion and spray drying typically result in NPs with a diameter greater than 300 nm and nanoprecipitation results in NPs with a diameter of 100-200 nm (Huang and Zhang, Biotechnol J 2018, 13(1)). Although high-pressure homogenization can be scaled-up to meet large production demands, factors including high volume of waste material, requirement of pre-emulsion due to phrase separation, and manual handling of liquids limit its applicability (Operti, et al., International Journal of Pharmaceutics 2018, 550(1), 140-148). In addition, these methods are not easily scalable to meet the manufacturing demands of clinical trials.

To address these limitations, platforms for the efficient production of delivery systems that effectively and selectively deliver therapeutic, diagnostic, and/or prophylactic agents in vivo are needed.

Therefore, it is an object of the invention to provide effective ways of delivering therapeutic, diagnostic, and/or prophylactic agents in vivo.

It is also an object of the invention to provide methods of making, and the resulting population, of nanoparticles that are suitable for in vivo delivery of therapeutic, diagnostic, and/or prophylactic agents, particularly polymeric nanoparticles that are taken up selectively by lung cells and/or bone marrow cells.

### SUMMARY OF THE INVENTION

Compositions containing populations of NPs that show selective uptake by cells of certain tissues, specifically lung cells and/or bone marrow cells, have been developed. Typically, the NPs show this uptake by virtue of their size and do not include targeting agent on the surface of the nanoparticles. Representative lung cells include type I alveolar epithelial cells and/or alveolar macrophage cells, while the bone marrow cells include hematopoietic stem and progenitor cells. NPs are formed of biocompatible polymers, most preferably biodegradable polymers. In a particularly preferred embodiment, the population of nanoparticles is formed of a polyester such as poly(lactic acid-co-glycolic acid), has a diameter between about 70 nm and about 220 nm, and at least 90% of the nanoparticles have a diameter between about 110 nm and about 129 nm. The nanoparticles can be used for delivery of therapeutic, prophylactic and/or diagnostic agents.

In a preferred embodiment, the NPS are used to deliver oligomers of peptide nucleic acids (PNAs) and donor DNAs (PNA/donor DNA) for gene editing technologies, typically with a loading between about 0.2 mg/mL and about 5 mg/mL, as measured by absorbance. The loading can also be expressed in terms of weight of the drug in nanoparticles to the weight of the nanoparticles, such as between about 0.1% wt/wt and about 10% wt/wt, as measured using a standard analytical method such as high-performance liquid chromatography. To achieve improved control of the sizes of the nanoparticles in the population, the nanoparticles are manufactured using a microfluidic system. By virtue of selective uptake by lung and/or bone marrow cells, the compositions can be used in the in vivo treatment of a variety of lung and blood disorders, in particular, disorders arising from genetic mutations affecting the lung and/or blood.

### BRIEF DESCRIPTION OF THE DRAWINGS

FIGS. 1A-1C are column and point graphs showing the effects of altering NanoAssemblr™ parameters on the sizes of PLGA NPs. The graphs show a comparison of NP size with changes in (i) flow rate ratio of aqueous to organic phase (FIG. 1A); (ii) stabilizer concentration (FIG. 1B); and (iii) PLGA polymer concentration (FIG. 1C).

FIGS. 2A and 2B are schematics of a formulation and characterization, respectively, of various sized DiD-loaded PLGA NPs. In FIG. 2A, PLGA NPs are formulated by injecting ACN, PLGA, 0.5 wt % DiD and 2% PVA into the NanoAssemblr™. NPs are washed in water immediately following formulation. FIG. 2B shows hydrodynamic diameter, PDI, and zeta-potential of NP formulations. Data are shown as mean±SD (n=3).

FIG. 3 is a column graphs showing quantification of average radians in whole organ ex vivo biodistribution of various sized PLGA NPs. Data are shown as mean±SEM (n=3). Statistical significance was calculated using a one-way ANOVA test and significance is represented on graphs as *p≤0.05. For each organ, the columns are, from left to right: control, NP-1, NP-2, NP-3, and NP-4.

FIGS. 4A-4E show in vivo biodistribution of various sized PLGA NPs in bulk tissue. FIG. 4A is a column graph showing normalized mean fluorescence intensity (nMFI) of DiD expression in all tissues quantified by flow cytometry. For each organ, the columns are, from left to right: control, NP-1, NP-2, NP-3, and NP-4. FIG. 4B shows representative flow cytometry histograms of DiD fluorescence in bulk lung tissue and FIG. 4C is a column graph showing the nMFI of DiD expression. FIG. 4D shows representative flow cytometry histograms of DiD fluorescence in bulk bone marrow and FIG. 4E is a column graph showing the nMFI of DiD expression. Data are shown as mean±SEM (n=3). Statistical significance was calculated using a one-way ANOVA test and significance is represented on graphs as *p≤0.05, ***p≤0.001, and ****p≤0.0001.

FIGS. 5A-5F show cell-specific association of PLGA NPs in lung tissue. Alveolar epithelial type I cells were gated on P2X7R⁺ and were found to represent 14.4% of the overall cell population; alveolar macrophages were gated on F480⁺ and were found to represent 17.7% of the overall cell population; and endothelial cells were gated on CD31⁺ and were found to represent 13.3% of the overall cell population FIGS. 5A-5C show representative histograms of DiD fluorescence in: P2X7R⁺ (FIG. 5A); F480⁺ (FIG. 5B); and CD31⁺ (FIG. 5C) cell populations isolated from lung tissue 24 h post-injection with NP-1, NP-2, NP-3, and NP-4. FIGS. 5D-5F are column graphs showing nMFI of: P2X7R⁺DiD⁺ (FIG. 5D); F480⁺ DiD⁺ (FIG. 5E); and CD31⁺DiD⁺ (FIG. 5F) cell populations at 24 h. Data are shown as mean±SEM (n=3). Statistical significance was calculated using a one-way ANOVA test and significance is represented on graphs as: not significant, ns, p>0.05; *p≤0.05; **p≤0.01; ***p≤0.001; and ****p≤0.0001.

FIGS. 6A-6C are column graphs showing the percentage of DiD⁺ cells in specific lung cell populations. Each graph shows a comparison of the percent of DiD⁺ cells in: P2X7R⁺ (FIG. 6A); F480⁺ (FIG. 6B); and CD31⁺ (FIG. 6C). Statistical significance was calculated using a one-way ANOVA test and significance is represented on graphs as: not significant, ns, p>0.05; *p≤0.05; and ***p≤0.001.

FIGS. 7A and 7B show cell-specific association of PLGA NPs in bone marrow hematopoietic stem and progenitor cells (HSPCs), as gated on CD117⁺. FIG. 7A is a representative histogram showing DiD fluorescence in isolated HPSCs 24 h post-injection with NP-1, NP-2, NP-3, and NP-4. FIG. 7B is the nMFI of HSPCs at 24 h. Data is shown as mean±SEM (n=3). Statistical significance was calculated using a one-way ANOVA test and significance is represented on graphs as ns p>0.05 and **p≤0.01.

FIG. 8 shows the percentage of DiD⁺ cells in the hematopoietic stem and progenitor cell population. Comparison of the percent DiD⁺ cells in CD117⁺ bone marrow cells. Statistical significance was calculated using a one-way ANOVA test and significance is represented on graphs as: not significant, ns, p>0.05 and **p≤0.01.

FIGS. 9A and 9B are column graphs showing the quantification of uptake, as nanoparticle intensity, of NP-1 and NP-2 in representative confocal images of bone marrow (FIG. 9A) and lung tissue (FIG. 9B) 24 h post administration, compared to untreated control. Data are shown as mean±SEM (n=3). Statistical significance was calculated using an unpaired t-test and significance is represented on graphs as *p≤0.05 and **p≤0.01.

FIG. 10 is a point graph showing in vivo gene editing in IVS2-654 β-thalassemic mice using stem cell factor and nanoparticles with hydrodynamic diameters of 120 nm and 300 nm.

FIGS. 11A-11E are point graphs showing in vivo gene editing in IVS2-654 β-thalassemic mice using stem cell factor and nanoparticles with hydrodynamic diameters of 120 nm, for different tissues: bone marrow (FIG. 11A), blood (FIG. 11B), lung (FIG. 11C), non-parenchymal cells of the liver (FIG. 11D), and hepatocytes (FIG. 11E).

### DETAILED DESCRIPTION OF THE INVENTION

### I. Definitions

The term “biodegradable,” generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The terms “effective amount” or “therapeutically effective amount” means a dosage sufficient to reduce or inhibit one or more symptoms of a disorder, disease, or condition being treated, or to otherwise provide a desired pharmacologic and/or physiologic effect. The precise dosage will vary according to a variety of factors such as subject-dependent variables (such as, age, immune system health, etc.), the severity of the disease or disorder being treated, as well as the route of administration and the pharmacokinetics of the agent being administered. The term “treating” refers to preventing or alleviating one or more symptoms of a disease, disorder, or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The term “pharmaceutically acceptable,” refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable carrier,” refers to all components of a pharmaceutical formulation which facilitate the delivery of the composition in vivo. Pharmaceutically acceptable carriers include, but are not limited to, diluents, preservatives, binders, lubricants, disintegrators, swelling agents, fillers, stabilizers, and combinations thereof.

“Nanoparticle” generally refers to a particle having a diameter, such as an average diameter, between about 10 nm up to but not including about 1 micron. The particles can have any shape. NPs having a spherical shape are generally referred to as “nanospheres.”

“Non-solvent,” “polymer non-solvent,” or “non-solvent of the polymer” are art-recognized terms, and are used interchangeably to refer to a “poor” solvent for a polymer, i.e., a solvent in which a polymer dissolves poorly.

“Parenteral administration” and “administered parenterally” are art-recognized terms, and include modes of administration other than enteral and topical administration, such as intra-venous, intra-muscular, intra-pleural, intra-vascular, intra-pericardial, intra-arterial, intra-thecal, intra-capsular, intra-orbital, intra-cardiac, intra-dennal, intra-peritoneal, trans-tracheal, subcutaneous, subcuticular, intra-articular, subcapsular, subarachnoid, intra-spinal and intra-stemal injection and infusion. “Enteral administration” refers to oral or other administration to the gastrointestinal tract.

The term “small molecule,” as used herein, generally refers to an organic molecule that is less than about 2000 g/mol in molecular weight, less than about 1500 g/mol, less than about 1000 g/mol, less than about 800 g/mol, or less than about 500 g/mol.

The term “targeting agent” refers to a chemical compound that can direct a NP to a receptor site on a selected cell or tissue type, can serve as an attachment molecule, or serve to couple or attach another molecule. The term “direct,” as relates to chemical compounds, refers to causing a NP to preferentially attach to a selected cell or tissue type. This targeting agent, generally binds to its receptor with high affinity and specificity.

### II. Compositions

Populations of NPs that show selective uptake by cells of certain tissues, such as lung cells and/or bone marrow cells, have been developed. The NPs do not include targeting ligands. The selective uptake is achieved passively by virtue of the sizes of the NPs and in the absence of a targeting agent on their surface. Formulations containing the populations of NPs and a pharmaceutically acceptable excipient are particularly suited for parental administration of therapeutic, diagnostic, and/or prophylactic agents (collectively “agents”).

A. Nanoparticles

In general, the population of NPs has diameters within a specified range, with a high percentage of the NPs having diameters within a subset of the specified range. A subset or all of the NPs contain therapeutic, diagnostic, and/or prophylactic agents, or a combination thereof. The NPs are formed of biocompatible polymers, preferably biodegradable polymers. In some forms, the biodegradable polymers are hydrophobic.

Suitable size ranges for the population of NPs; (ii) polymers; and (iii) therapeutic, diagnostic, and/or prophylactic agents and loadings thereof, of these NPs are described below. Combinations of each of these sizes, polymers, agents, and loadings described below are specifically. The NPs provide controlled release of the therapeutic, diagnostic, and/or prophylactic agents. Preferably, the NPs are formed by utilizing a microfluidic platform. An exemplary microfluidic platform is the NanoAssemblr™ from Precision NanoSystems Inc.

i. Size and Polydispersity Index

Selective uptake of the population of NPs can be achieved passively by virtue of the sizes of the NPs and in the absence of a targeting agent on their surface.

In some forms, the population of NPs have a diameter between about 50 nm and about 350 nm, between about 70 nm and about 300 nm, or between about 70 nm and about 220 nm, and at least 85% of the NPs have a diameter selected from the range of between about 120 nm and about 145 nm, between about 125 nm and about 140 nm, between about 100 nm and about 135 nm, or between about 110 nm and about 129 nm.

In some forms, the population of NPs have a diameter between about 50 nm and about 350 nm, and at least 85% of the NPs have a diameter between about 120 nm and about 145 nm, between about 125 nm and about 140 nm, between about 100 nm and about 135 nm, or between about 110 nm and about 129 nm.

In some forms, the population of NPs have a diameter between about 70 nm and about 220 nm to 300 nm, and at least 85% of the NPs have a diameter between about 120 nm and about 145 nm, between about 125 nm and about 140 nm, between about 100 nm and about 135 nm, or between about 110 nm and about 129 nm.

In some forms, the population of NPs have a diameter between about 50 nm and about 350 nm, between about 70 nm and about 300 nm, or between about 70 nm and about 220 nm, and at least 90% of the NPs have a diameter between about 120 nm and about 145 nm, between about 125 nm and about 140 nm, between about 100 nm and about 135 nm, or between about 110 nm and about 129 nm.

In some forms, the population of NPs have a diameter between about 70 nm and about 300 nm, and at least 90% of the NPs have a diameter selected from the range of between about 120 nm and about 145 nm, between about 125 nm and about 140 nm, between about 100 nm and about 135 nm, or between about 110 nm and about 129 nm.

In some forms, the NPs have a polydispersity index less than 0.25.

ii. Polymers

The NPs can be formed of one or more biocompatible polymers, preferably biodegradable polymers. The polymers can be hydrophobic, hydrophilic, or amphiphilic polymers that can be broken down hydrolytically or enzymatically in vitro or in vivo. The polymers can be soluble polymers crosslinked by hydrolysable cross-linking groups to render the crosslinked polymer insoluble or sparingly soluble in water. Exemplary polymers are discussed below. Copolymers such as random, block, or graft copolymers, or blends of the polymers listed below can also be used.

The weight average molecular weight can vary for a given polymer but is generally between about 1000 Daltons and 1,000,000 Daltons, between about 1000 Daltons and about 500,000 Dalton, between about 1000 Daltons and about 250,000 Daltons, between about 1000 Daltons and about 100,000 Daltons, between about 5,000 Daltons and about 100,000 Daltons, between about 5,000 Daltons and about 75,000 Daltons, between about 5,000 Daltons and about 50,000 Daltons, or between about 5,000 Daltons and about 25,000 Daltons.

1. Hydrophobic Polymers

The NPs can formed of one or more hydrophobic polymers. In some forms, the hydrophobic polymers are biodegradable. Examples of suitable hydrophobic polymers include polyesters such as polyhydroxy acids (such as poly(lactic acid-co-glycolic acid)s, poly(lactic acid), poly(glycolic acid)), polycaprolactones, polyhydroxyalkanoates (such as poly-3-hydroxybutyrate, poly4-hydroxybutyrate, polyhydroxyvalerates), poly(lactide-co-caprolactones); poly(anhydride)s; poly(orthoester)s, as well as copolymers thereof.

In some forms, the hydrophobic polymers include polyesters such as polyhydroxy acids (such as poly(lactic acid-co-glycolic acid)s, poly(lactic acid), poly(glycolic acid)), polycaprolactones, polyhydroxyalkanoates (such as poly-3-hydroxybutyrate, poly4-hydroxybutyrate, polyhydroxyvalerates), poly(lactide-co-caprolactones); poly(anhydride)s; poly(orthoester)s; poly(beta-amino ester)s; poly(amine-co-ester)s; poly(amine-co-ester-co-ortho ester)s, and copolymers thereof.

In a preferred embodiment, the hydrophobic polymer is a polyester, most preferably a polyhydroxy acid such as poly(lactic acid-co-glycolic acid), poly(lactic acid), or poly(glycolic acid).

2. Hydrophilic Polymers

The NPs can contain one or more hydrophilic polymers. Preferably, the hydrophilic polymers are biodegradable. Hydrophilic polymers include polyalkylene glycol such as polyethylene glycol (PEG); polysaccharides such as cellulose and starch and derivatives thereof; hydrophilic polypeptides such as poly-L-glutamic acid, gamma-polyglutamic acid, poly-L-aspartic acid, poly-L-serine, or poly-L-lysine; poly(oxyethylated polyol); poly(olefinic alcohol) such as poly(vinyl alcohol); poly(vinylpyrrolidone); poly(N-hydroxyalkyl methacrylamide) such as poly(N-hydroxyethyl methacrylamide); poly(N-hydroxyalkyl methacrylate) such as poly(N-hydroxyethyl methacrylate); hydrophilic poly(hydroxy acids); and copolymers thereof. In some forms, the hydrophilic polymer is a polyalkylene glycol such as PEG or a poloxamer

3. Amphiphilic Polymers

The NPs can contain one or more amphiphilic polymers, preferably biodegradable amphiphilic polymers. The amphiphilic polymers contain a hydrophobic polymer portion and a hydrophilic polymer portion. The hydrophobic polymer portion and hydrophilic polymer portion can include any of the hydrophobic polymers and hydrophilic polymers, respectively, described above. In a non-limiting example, the hydrophobic polymer portion is a polymer formed from a polyester such as polyhydroxy acids (such as poly(lactic acid), poly(glycolic acid), and poly(lactic acid-co-glycolic acid)s), polycaprolactones, polyhydroxyalkanoates (such as poly-3-hydroxybutyrate, poly4-hydroxybutyrate, polyhydroxyvalerates), poly(lactide-co-caprolactones); poly(anhydride)s; poly(orthoester)s; and hydrophobic polyethers (such as polypropylene glycol); as well as copolymers thereof. The hydrophilic polymer portion can contain a polymer such as a polyalkylene oxide such as polypropylene glycol or polyethylene glycol (PEG); polysaccharides such as cellulose and starch; hydrophilic polypeptides such as poly-L-glutamic acid, gamma-polyglutamic acid, poly-L-aspartic acid, poly-L-serine, or poly-L-lysine; poly(oxyethylated polyol); poly(olefinic alcohol) such as poly(vinyl alcohol); poly(vinylpyrrolidone); polyacrylamides or polymethaacrylamides including poly(N-hydroxyalkyl methacrylamides) such as poly(N-hydroxyethyl methacrylamide); poly(N-hydroxyalkyl methacrylates) such as poly(N-hydroxyethyl methacrylate); hydrophilic poly(hydroxy acids); and copolymers thereof. Examples of amphiphilic polymers that can be generated from this group include polyester-PEG copolymers such as poly(lactic acid-co-glycolic acid)-PEG (PLGA-PEG), poly(lactic acid)-PEG (PLA-PEG), poly(glycolic acid)-PEG (PGA-PEG), and polycaprolactone-PEG (PCL-PEG); and hydrophobic polyethers-PEG, such as polypropylene glycol-PEG (PPG-PEG), PEG-PPG-PEG, PPG-PEG-PPG.

iii. Therapeutic, Diagnostic, and Prophylactic Agents, and Loading

The population of NPs is useful for carrying, presenting, and/or delivering therapeutic, diagnostic, or prophylactic agents. A subset or all of the NPs contain one or more of these agents. The agents can be covalently or non-covalently conjugated to a polymer or other component of the NPs. Each of these agents can be associated with the surface of the NPs, encapsulated within the NPs, and/or dispersed throughout a matrix of polymers of the NPs.

In some forms, the agents can be, independently, nucleic acids, proteins, peptides, lipids, polysaccharides, small molecules, or a combination thereof.

In some forms, the agent is one or more nucleic acids. The nucleic acid can alter, correct, or replace an endogenous nucleic acid sequence. The nucleic acid can be used to, for example, treat diseases of the lung (such as cystic fibrosis, alpha-1 antitrypsin deficiency, idiopathic pulmonary fibrosis, etc.), blood disorders (such as sickle cell disease, thalassemia, Kostmann syndrome, Schwachman-Diamond syndrome, hemophilia, von Willebrand disease, platelet function disorders, thrombocytopenia, and hypofibrinogenemia and dysfibrinogenemia), and correct defects in genes via gene therapy. Gene therapy is a technique for correcting defective genes responsible for disease development. WO2018/187493 by Saltzman, et al., provides extensive details on gene therapy technologies, the contents of which are herein incorporated by reference. In some forms, the agent (such as nucleic acid) can be selected from peptide nucleic acids (PNAs), antisense DNAs and RNAs, DNAs coding for proteins, mRNAs, miRNAs, piRNAs, siRNAs, and combinations thereof. In some forms, the agent (such as nucleic acid) includes a combination of PNAs and donor DNAs. In some forms, the agents (such as nucleic acids) are oligonucleotides. In some forms, nucleic acid (such as PNA) and/or donor DNA (such as donor DNA oligonucleotide) can be incorporated the same NP or separately in different NPs. For example, PNA and donor DNA can be mixed and packaged together in a NP. In some forms, PNA and donor DNA can be formulated in different compositions and packaged separately into separate NPs wherein the NPs are similarly or identically composed and/or manufactured. In some forms, the PNA and donor DNA are packaged separately into separate NPs wherein the NPs are differentially composed and/or manufactured.

1. Gene Editing Technology

In some forms, the therapeutic, diagnostic, and/or prophylactic agents are, or encode, a gene editing technology. Gene editing technologies can be used alone or in combination with a potentiating agent and/or other agents. Exemplary gene editing technologies include, but are not limited to, triplex-forming, pseudocomplementary oligonucleotides, CRISPR/Cas, zinc finger nucleases, TALENs, and small fragment homologous replacement. WO2018/187493 by Saltzman, et al., provides extensive details on gene therapy technologies, the contents of which are herein incorporated by reference. The gene editing composition can be a pseudocomplementary oligonucleotide or PNA oligomer.

Triplex-Forming Molecules (TFMs)

a. Compositions

Compositions containing “triplex-forming molecules,” that bind to duplex DNA in a sequence-specific manner to form a triple-stranded structure include, but are not limited to, triplex-forming oligonucleotides (TFOs), peptide nucleic acids (PNA), and “tail clamp” PNA (tcPNA). The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids (PNAs). Triplex forming molecules are described in U.S. Pat. Nos. 5,962,426, 6,303,376, 7,078,389, 7,279,463, 8,658,608, U.S. Published Application Nos. 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, Rogers, et al., Proc Natl Acad Sci USA, 99:16695-16700 (2002), Majumdar, et al., Nature Genetics, 20:212-214 (1998), Chin, et al., Proc Natl Acad Sci USA, 105:13514-13519 (2008), and Schleifman, et al., Chem Biol., 18:1189-1198 (2011). Donor oligonucleotides can include one or more phosphorothioate linkages.

### Triplex-Forming Oligonucleotides

Triplex-forming oligonucleotides (TFOs) are defined as oligonucleotides which bind as third strands to duplex DNA in a sequence specific manner Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The nucleobase (sometimes referred to herein simply as “base”) composition may be homopurine or homopyrimidine. Alternatively, the nucleobase composition may be polypurine or polypyrimidine. However, other compositions are also useful.

Preferably, the oligonucleotide/oligomer binds to or hybridizes to the target sequence under conditions of high stringency and specificity. Most preferably, the oligonucleotides/oligomers bind in a sequence-specific manner within the major groove of duplex DNA. An oligonucleotide substantially complementary, based on the third strand binding code, to the target region of the double-stranded nucleic acid molecule is preferred.

As used herein, a triplex forming molecule is said to be substantially complementary to a target region when the oligonucleotide has a nucleobase composition which allows for the formation of a triple-helix with the target region.

### Peptide Nucleic Acids

In some forms, the triplex-forming molecules are peptide nucleic acids (PNAs). PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules (see Bentin, et al., Nucl. Acids Res., 34(20): 5790-5799 (2006) and Hansen, et al., Nucl. Acids Res., 37(13): 4498-4507 (2009)), or two PNA molecules linked together by a linker of sufficient flexibility to form a single bis-PNA molecule (See: U.S. Pat. No. 6,441,130).

Suitable molecules for use in linkers of bis-PNA molecules include, but are not limited to, 8-amino-2, 6, 10-trioxaoctanoic acid, 8-amino-3,6-dioxaoctanoic acid, and 6-aminohexanoic acid. In some embodiments, these molecules are referred to an O-linker, and can be represented by “O” in the sequences presented herein. Poly(ethylene) glycol monomers can also be used in bis-PNA linkers. A bis-PNA linker can contain multiple linker residues in any combination of two or more of the foregoing.

PNAs can also include other positively charged moieties to increase the solubility of the PNA and increase the affinity of the PNA for duplex DNA. Commonly used positively charged moieties include the amino acids lysine and arginine (such as, as additional substituents attached to the C- or N-terminus of the PNA oligomer (or a segment thereof) or as a side-chain modification of the backbone (see Huang, et al., Arch. Pharm. Res. 35(3): 517-522 (2012) and Jain, et al., JOC, 79(20): 9567-9577 (2014)), although other positively charged moieties may also be useful (See for Example: U.S. Pat. No. 6,326,479). In some forms, the PNA oligomer can have one or more ‘miniPEG’ side chain modifications of the backbone (see, for example, U.S. Pat. No. 9,193,758 and Sahu, et al., JOC, 76: 5614-5627 (2011)).

### Tail Clamp Peptide Nucleic Acids

Although polypurine:polypyrimidine stretches do exist in mammalian genomes, it can be desirable to target triplex formation in the absence of this requirement. In some forms such as PNA, triplex-forming molecules include a “tail” added to the end of the Watson-Crick binding portion. The tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99(26):16695-700 (2002)).

Tails added to clamp PNAs (sometimes referred to as bis-PNAs) form tail-clamp PNAs (referred to as tcPNAs) that have been described by Kaihatsu, et al., Biochemistry, 42(47):13996-4003 (2003); Bentin, et al., Biochemistry, 42(47):13987-95 (2003).

In some forms a tcPNA system contains:

a) optionally, a positively charged region having a positively charged amino acid subunit, such as, a lysine subunit;

b) a first region comprising a plurality of PNA subunits having Hoogsteen homology with a target sequence;

c) a second region comprising a plurality of PNA subunits having Watson Crick homology binding with the target sequence;

d) a third region comprising a plurality of PNA subunits having Watson Crick homology binding with a tail target sequence;

e) optionally, a second positively charged region having a positively charged amino acid subunit, such as, a lysine subunit.

In some forms, a linker is disposed between b) and c). In some forms, one or more PNA monomers of the tail claim is modified as disclosed herein.

### PNA Modifications

PNAs can also include other positively charged moieties to increase the solubility of the PNA and increase the affinity of the PNA for duplex DNA. Common modifications to PNA are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to DNA to generate a chimeric oligomer, redesign of the backbone architecture, conjugation of PNA to DNA or RNA. These modifications improve solubility but often result in reduced binding affinity and/or sequence specificity. In some forms, the some or all of the PNA residues are modified at the gamma position in the polyamide backbone (γPNAs) as illustrated below (wherein “B” is a nucleobase and “R” is a substitution at the gamma position).

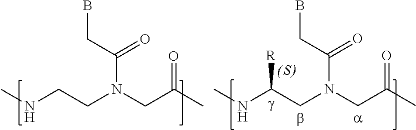

One class of γ substitution, is miniPEG, but other residues and side chains can be considered, and even mixed substitutions can be used to tune the properties of the oligomers. “MiniPEG” and “MP” refers to diethylene glycol. In the some forms, some or all of the PNA residues are miniPEG-containing γPNAs (Sahu, et al., J. Org. Chem., 76, 5614-5627 (2011). In some forms, tcPNAs are prepared wherein every other PNA residue on the Watson-Crick binding side of the linker is a miniPEG-containing γPNA. Accordingly, for these forms, the tail clamp side of the PNA has alternating classic PNA and miniPEG-containing γPNA residues.

Additionally, any of the triplex forming sequences can be modified to include guanidine-G-clamp (“G-clamp”) PNA residues(s) to enhance PNA binding, wherein the G-clamp is linked to the backbone as any other nucleobase would be.

In some forms, the gene editing composition includes at least one PNA oligomer. The at least one PNA oligomer can be a modified PNA oligomer including at least one modification at a gamma position of a backbone carbon. The modified PNA oligomer can include at least one miniPEG modification at a gamma position of a backbone carbon. The gene editing composition can include at least one donor oligonucleotide.

The PNA can include a Hoogsteen binding peptide nucleic acid (PNA) segment and a Watson-Crick binding PNA segment collectively totaling no more than 50 nucleobases in length, wherein the two segments bind or hybridize to a target region of a genomic DNA comprising a polypurine stretch to induce strand invasion, displacement, and formation of a triple-stranded composition among the two PNA segments and the polypurine stretch of the genomic DNA, wherein the Hoogsteen binding segment binds to the target region by Hoogsteen binding for a length of least five nucleobases, and wherein the Watson-Crick binding segment binds to the target region by Watson-Crick binding for a length of least five nucleobases.

The PNA segments can include a gamma modification of a backbone carbon. The gamma modification can be a gamma miniPEG modification. The Hoogsteen binding segment can include one or more chemically modified cytosines selected from the group consisting of pseudocytosine, pseudoisocytosine, and 5-methylcytosine. The Watson-Crick binding segment can include a sequence of up to fifteen nucleobases that binds to the target duplex by Watson-Crick binding outside of the triplex. The two segments can be linked by a linker. In some forms, all of the peptide nucleic acid residues in the Hoogsteen-binding segment only, in the Watson-Crick-binding segment only, or across the entire PNA oligomer include a gamma modification of a backbone carbon. In some forms, one or more of the peptide nucleic acid residues in the Hoogsteen-binding segment only or in the Watson-Crick-binding segment only of the PNA oligomer include a gamma modification of a backbone carbon. In some forms, alternating peptide nucleic acid residues in the Hoogsteen-binding portion only, in the Watson-Crick-binding portion only, or across the entire PNA oligomer include a gamma modification of a backbone carbon.

In some forms, least one gamma modification of the backbone carbon is a gamma miniPEG modification. In some forms, at least one gamma modification is a side chain of an amino acid selected from the group consisting of alanine, serine, threonine, cysteine, valine, leucine, isoleucine, methionine, phenylalanine, tyrosine, aspartic acid, glutamic acid, asparagine, glutamine, histidine, lysine, arginine, and the derivatives thereof. In some forms, all gamma modifications are gamma miniPEG modifications. Optionally, at least one PNA segment contains a clamp-G (9-(2-guanidinoethoxy) phenoxazine).

b. Triplex-Forming Target Sequence Considerations

The triplex-forming molecules bind to a predetermined target region referred to herein as the “target sequence,” “target region,” or “target site.” The target sequence for the triplex-forming molecules can be within or adjacent to a human gene encoding, for example the beta globin, cystic fibrosis transmembrane conductance regulator (CFTR) or other gene discussed in more detail below, or an enzyme necessary for the metabolism of lipids, glycoproteins, or mucopolysaccharides, or another gene in need of correction. The target sequence can be within the coding DNA sequence of the gene or within an intron. The target sequence can also be within DNA sequences which regulate expression of the target gene, including promoter or enhancer sequences or sites that regulate RNA splicing.

The nucleotide sequences of the triplex-forming molecules are selected based on the sequence of the target sequence, the physical constraints, and the preference for a low dissociation constant (K_{d}) for the triplex-forming molecules/target sequence. As used herein, triplex-forming molecules are said to be substantially complementary to a target region when the triplex-forming molecules has a nucleobase composition which allows for the formation of a triple-helix with the target region. A triplex-forming molecule can be substantially complementary to a target region even when there are non-complementary nucleobases present in the triplex-forming molecules.

There are a variety of structural motifs available which can be used to determine the nucleotide sequence of a substantially complementary oligonucleotide. Preferably, the triplex-forming molecules bind to or hybridize to the target sequence under conditions of high stringency and specificity. Reaction conditions for in vitro triple helix formation of an triplex-forming molecules probe or primer to a nucleic acid sequence vary from triplex-forming molecules to triplex-forming molecules, depending on factors such as the length triplex-forming molecules, the number of G:C and A:T base pairs, and the composition of the buffer utilized in the hybridization reaction.

### Target Sequence Considerations for TFOs

Preferably, the TFO is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. Most preferably, the oligonucleotides bind in a sequence-specific manner within the major groove of duplex DNA. An oligonucleotide substantially complementary, based on the third strand binding code, to the target region of the double-stranded nucleic acid molecule is preferred. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

TFOs are preferably generated using known DNA and/or PNA synthesis procedures. In some forms, oligonucleotides are generated synthetically. Oligonucleotides can also be chemically modified using standard methods that are well known in the art.

### Target Sequence Considerations for PNAs

Some triplex-forming molecules, such as PNA, PNA clamps and tail clamp PNAs (tcPNAs) invade the target duplex, with displacement of the polypyrimidine strand, and induce triplex formation with the polypurine strand of the target duplex by both Watson-Crick and Hoogsteen binding. Preferably, both the Watson-Crick and Hoogsteen binding portions of the triplex forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleobase-containing residues in length. The Watson-Crick portion should be 9 or more nucleobase-containing residues in length, optionally including a tail sequence. More preferably, the Watson-Crick binding portion is between about 9 and about 30 nucleobase-containing residues in length, optionally including a tail sequence of between 0 and about 15 nucleobase-containing residues. More preferably, the Watson-Crick binding portion is between about 10 and about 25 nucleobase-containing residues in length, optionally including a tail sequence of between 0 and about 10 nucleobase-containing residues in length. Preferably, the Watson-Crick binding portion is between 15 and 25 nucleobase-containing residues in length, optionally including a tail sequence of between 5 and 10 nucleobase-containing residues in length. The Hoogsteen binding portion should be 6 or more nucleobase residues in length. Most preferably, the Hoogsteen binding portion is between about 6 and 15 nucleobase-containing residues in length, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobase-containing residues, known as a “tail,” to the Watson-Crick binding portion of the triplex-forming molecules allows the Watson-Crick binding portion to bind/hybridize to the target strand outside the site of polypurine sequence for triplex formation. These additional bases further reduce the requirement for the polypurine:polypyrimidine stretch in the target duplex and therefore increase the number of potential target sites. Triplex-forming molecules (TFMs) including, such as, triplex-forming oligonucleotides (TFOs) and helix-invading peptide nucleic acids (bis-PNAs and tcPNAs), also generally utilize a polypurine:polypyrimidine sequence to a form a triple helix. Traditional nucleic acid TFOs may need a stretch of at least 15 and preferably 30 or more nucleobase-containing residues. Peptide nucleic acids need fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide. Methods of making the triplex-forming molecules are known in the art.

2. Donor Oligonucleotides

In some forms, the gene editing composition includes or is administered in combination with a donor oligonucleotide. The donor oligonucleotide can be encapsulated or entrapped in the same or different NPs from other agents such as the triplex forming composition.

a. Preferred Donor Oligonucleotide Design for Triplex and Double-Duplex Based Technologies

The triplex forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence targets a region between 0 to 800 bases from the target binding site of the triplex-forming molecules. More preferably the donor oligonucleotide sequence targets a region between 25 to 75 bases from the target binding site of the triplex-forming molecules. Most preferably that the donor oligonucleotide sequence targets a region about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In some forms the oligonucleotide donor is between 25 and 80 nucleobases. In another form, the non-tethered donor oligonucleotide is about 50 to 60 nucleotides in length.

The donor oligonucleotides contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences or sequences that regulate RNA splicing.

Compositions including triplex-forming molecules such as tcPNA may include one or more than one donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic nucleobases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. One exemplary modification is a thiophosphate ester linkage. Modifications to the donor oligonucleotide should not prevent the donor oligonucleotide from successfully recombining at the recombination target sequence in the presence of triplex-forming molecules.

b. Preferred Donor Oligonucleotides Design for Nuclease-Based Technologies

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some forms, the genome editing composition optionally includes a donor oligonucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence by cleaving the target DNA sequence and allowing the cell to repair the sequence in the absence of an exogenously provided donor polynucleotide. Alternatively, if the genome editing composition includes a donor oligonucleotide sequence that includes at least a segment with homology to the target DNA sequence, the methods can be used to add, i.e., insert or replace, nucleic acid material to a target DNA sequence (such as, to “knock in” a nucleic acid that encodes for a protein, an siRNA, an miRNA, etc.), to add a tag (such as, 6×His, a fluorescent protein (such as, a green fluorescent protein; a yellow fluorescent protein, etc.), hemagglutinin (HA), FLAG, etc.), to add a regulatory sequence to a gene (such as, promoter, polyadenylation signal, internal ribosome entry sequence (IRES), 2A peptide, start codon, stop codon, splice signal, localization signal, etc.), to modify a nucleic acid sequence (such as, introduce a mutation), and the like. As such, the compositions can be used to modify DNA in a site-specific, i.e., “targeted”, way, for example gene knock-out, gene knock-in, gene editing, gene tagging, etc. as used in, for example, gene therapy.

In applications in which it is desirable to insert an oligonucleotide sequence into a target DNA sequence, an oligonucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, such as, 70%, 80%, 85%, 90%, 95%, or 100% homology with the nucleotide sequences flanking the cleavage site, such as, within about 50 bases or less of the cleavage site, such as, within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some forms, the donor oligonucleotide includes a non-homologous sequence flanked by two regions of homology, such that homology-directed repair between the target DNA region and the two flanking sequences results in insertion of the non-homologous sequence at the target region.

The agents (therapeutic, diagnostic, and/or prophylactic agents) can have a loading between about 0.2 mg/mL and about 5 mg/mL, between about 0.2 mg/mL and about 2 mg/mL, between about 0.2 mg/mL and about 1 mg/mL, as measured by absorbance. In some forms, the loading of the agents can also be expressed in terms of weight of the drug in nanoparticles to the weight of the nanoparticles. In these forms, agents can have a loading between about 0.1% wt/wt and about 20% wt/wt, between 0.1% wt/wt and about 15% wt/wt, between about 0.1% wt/wt and about 10% wt/wt, as measured using a standard analytical method such as high-performance liquid chromatography, gas chromatography-mass spectrometry, or liquid chromatography-mass spectrometry.

In a particularly preferred embodiment, the population of NPs have a diameter between about 70 nm and about 220 nm, with at least 90% of the NPs having a diameter between about 110 nm and about 129 nm. In this preferred embodiment, the therapeutic, diagnostic, and/or prophylactic agents contain gene editing technology, particularly peptide nucleic acids (PNAs) and donor DNAs, i.e., PNA/donor DNA, and preferably oligomers thereof. Preferably, the PNA/donor DNA have a loading between about 0.2 mg/mL and about 5 mg/mL, as measured by absorbance. Preferably, the NPs contain a hydrophobic polymer, and preferably poly(lactic acid-co-glycolic acid) (PLGA).

B. Formulations

Pharmaceutical compositions containing the population of NPs can be formulated for parenteral administration. The formulations are designed according to the route of administration and can be formulated in dosage forms appropriate for each route of administration. The NPs and pharmaceutical compositions are typically administered by intravenous, or subcutaneous, intramuscular injection or intranasal or pulmonary formulations. They may also be fabricated for oral delivery, if delivered in an enteric capsule.

The formulation can be in the form of a suspension or emulsion. In general, pharmaceutical compositions are provided including effective amounts of the therapeutic, diagnostic, and/or prophylactic agents and optionally include pharmaceutically acceptable diluents, preservatives, solubilizers, emulsifiers, and/or carriers. Such compositions include sterile water, buffered saline of various buffer content (such as, Tris HCl, acetate, phosphate), pH and ionic strength; and optionally, additives such as detergents and solubilizing agents (such as, TWEEN® 20, TWEEN® 80 also referred to as polysorbate 20 or 80), anti-oxidants (such as, ascorbic acid, sodium metabisulfite), and preservatives. Preferably, the suspension or emulsion include water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. Examples of non-aqueous solvents or vehicles are propylene glycol, polyethylene glycol, vegetable oils, such as olive oil and corn oil, gelatin, and injectable organic esters such as ethyl oleate.

In some forms, for example when the target cells are lung cells, the population of NPs and pharmaceutical compositions thereof can be formulated for pulmonary administration. The administration can include delivery of the composition to the lungs or nasal mucosa.

The term aerosol as used herein refers to any preparation of a fine mist of particles, which can be in emulsion or a suspension, whether or not it is produced using a propellant. Aerosols can be produced using standard techniques, such as ultrasonication or high-pressure treatment.

Carriers for pulmonary formulations can be divided into those for dry powder formulations and for administration as emulsion or suspension. Aerosols for the delivery of therapeutic agents to the respiratory tract are known in the art. For administration via the upper respiratory tract, the formulation can be formulated into an emulsion or a suspension containing an aqueous component, such as, water or isotonic saline, buffered or un-buffered, or as a suspension, for intranasal administration as drops or as a spray. One skilled in the art can readily determine a suitable saline content and pH for an innocuous emulsion or a suspension for nasal and/or upper respiratory administration.

The formulations may be lyophilized and redissolved or resuspended immediately before use. The formulation may be sterilized by, for example, filtration through a bacteria retaining filter, by incorporating sterilizing agents into the compositions, by irradiating the compositions, or by heating the compositions.

### III. Methods of Making and Reagents Therefor

Preferably, the population of NPs is produced using a microfluidic system. Methods of making particles using microfluidics are known in the art. Suitable methods include those described in U.S. Patent Application Publication 2010/0022680 A1 by Karnik, et al. In general, the microfluidic device contains at least two channels that converge into a mixing apparatus. The channels are typically formed by lithography, etching, embossing, or molding of a polymeric surface. A source of fluid is attached to each channel, and the application of pressure to the source causes the flow of the fluid in the channel. The pressure may be applied by a syringe, a pump, and/or gravity, etc. The inlet streams of solutions with polymer; therapeutic, diagnostic, and/or prophylactic agents; payload; etc. converge and mix, and the resulting mixture is combined with a polymer non-solvent solution to form NPs having the desired size and loading.

In some forms, the therapeutic, diagnostic, and/or prophylactic agents are in the same inlet stream of the microfluidic system. In these forms, the inlet stream can be an organic phase or an aqueous phase.

In some forms, the therapeutic, diagnostic, and/or prophylactic agents are in separate inlet streams of the microfluidic system. In these forms, the inlet stream containing the therapeutic, diagnostic, and/or prophylactic agents can be an organic phase and the polymer in an aqueous phase. Alternatively, in these forms, the inlet stream containing the therapeutic, diagnostic, and/or prophylactic agents can be an aqueous phase and the polymer in an organic phase.

By varying the pressure and flow rate in the inlet channels and the nature and composition of the fluid sources NPs can be produced having reproducible size and structure.

In some forms, the flow rate ratio of the aqueous phase to organic phase is between 1:10 and 10:1, inclusive, between 1:5 and 5:1, inclusive, or between 1:1 and 5:1, inclusive.

In some forms, the flow rates of the organic and aqueous phases are independently between 1 mL/min and 20 mL/min, inclusive, 1 mL/min and 15 mL/min, inclusive, or between 2 mL/min and 14 mL/min, inclusive.

In some forms, the formulation volumes of the organic and aqueous phases are independently between 1 mL and 10 mL, inclusive, between 1 mL and 8 mL, inclusive, between 1 mL and 6 mL, inclusive, between 2 mL and 10 mL, inclusive, between 2 mL and 8 mL, inclusive, or between 2 mL and 6 mL, inclusive.

In some forms, the concentration of the aqueous phase is between 0.1% w/v and 5% w/v, inclusive, between 0.1% w/v and 4% w/v, inclusive, between 0.5% w/v and 5% w/v, inclusive, or between 0.5% w/v and 4% w/v, inclusive.

In some forms, the concentration of the organic phase can be between 1 mg/mL and 250 mg/mL, inclusive, between 1 mg/mL and 230 mg/mL, inclusive, between 1 mg/mL and 200 mg/mL, inclusive, between 2 mg/mL and 250 mg/mL, inclusive, between 2 mg/mL and 230 mg/mL, inclusive, between 2 mg/mL and 200 mg/mL, inclusive, between 5 mg/mL and 250 mg/mL, inclusive, between 5 mg/mL and 230 mg/mL, inclusive, or between 5 mg/mL and 200 mg/mL, inclusive.

In some forms, the flow rate ratio of the aqueous phase to organic phase is between 1:1 and 5:1, inclusive. In some forms, the flow rates of the organic and aqueous phases are between 2 mL/min and 14 mL/min. In some forms, the formulation volumes of the organic of the organic and aqueous phases are independently between 2 mL and 6 mL, inclusive. In some forms, the concentration of the aqueous phase is between 0.5% w/v and 4% w/v. In some forms, the concentration of the organic phase can be between 5 mg/mL and 200 mg/mL, inclusive.

Preferably, for these values of flow rate ratios, flow rates, formulation volumes, and/or concentration, the polymer in the organic phase contains a hydrophobic polymer such as PLGA, PLA, or PGA, preferably having a molecular weight between 30 kDa and 60 kDa, inclusive, (such as between 33 kDa and 55 kDa, inclusive).

In some forms, and as detailed in the Example section, the population of NPs is formed by providing a first fluid containing the biodegradable polymer, and contacting the fluid with a second fluid containing a non-solvent of the biodegradable polymer to produce the population of NPs. The first fluid may be miscible or immiscible with the second fluid containing the non-solvent of the biodegradable polymer. For example, as is discussed in the examples, a water-miscible liquid such as acetonitrile (ACN) may contain the biodegradable polymer, and NPs are formed as the ACN is contacted with water, a non-solvent of the biodegradable polymer, such as, by injecting or providing both fluids in separate channels in a microfluidic system at a flow rate ratio, controlled flow rate, formulation volume, and/or concentration, such that both fluids contact each other downstream. In some forms, the therapeutic, diagnostic, and/or prophylactic agents to be delivered are included in the fluid that contains the biodegradable polymer. The biodegradable polymer contained within the organic solvent or solution, upon contact with the non-solvent of the biodegradable polymer, may then precipitate to form a population of NPs, as described herein.

### IV. Methods of Using

Methods of using the compositions are provided, particularly for include delivery of one or more therapeutic, prophylactic, and/or diagnostic agents to lung cells for the treatment of lung disorders, and/or to bone marrow cells for the treatment of blood disorders. The methods typically include administering a subject in a need thereof an effective amount of a composition including therapeutic, diagnostic, and/or prophylactic agents encapsulated the NPs, wherein the NP contain biodegradable polymers. Preferred routes of administration include intra-venous injection or intranasal administration, if desired, for pulmonary formulations.

Lung disorders that can be treated include cystic fibrosis, alpha-1 antitrypsin deficiency, idiopathic pulmonary fibrosis, etc. Blood disorders that can be treated include: red blood cell disorders (sickle cell disease, thalassemia, hemolytic disease of the newborn, hemolytic anemia, spherocytosis, hemochromatosis, congenital sideroblastic anemia, congenital dyserythropoietic anemia, megaloblastic anemia (including pernicious anemia); white blood cell disorders (severe congenital neutropenia (Kostmann syndrome), cyclical neutropenia, chronic granulomatous disease, leukocyte adhesion deficiency, myeloperoxidase deficiency); bone marrow failure syndromes (aplastic anemia, congenital amegakaryocytic thrombocytopenia, diamond-Blackfan anemia, Fanconi anemia, Schwachman-Diamond syndrome, thrombocytopenia absent radius); bleeding disorders (hemophilia, von Willebrand disease, platelet function disorders, thrombocytopenia, hypofibrinogenemia and dysfibrinogenemia); thrombosis and anticoagulation disorders (thrombosis, Factor V Leiden, prothrombin gene mutation, protein C deficiency, protein S deficiency, antithrombin deficiency); and polycythemia vera.

The composition can also be used in gene therapy technologies to deliver therapeutic, diagnostic, and/or prophylactic agents in the in vivo treatment of one or more of the blood or lung disorders described above. The composition can be administered in vivo to a fetus, embryo, or to the mother, or other subject of appropriate age in need of treatment.

In some forms, the therapeutic agents include a donor nucleotide and optionally triplex-forming sequences. Exemplary triplex-forming sequences and donor nucleotides for some lung and blood disorders are described below.

A. Blood Disorders

i. Triplex-Forming Sequences

1. Beta Thalassemia

Exemplary triplex forming molecule and donor sequences, are provided in, for example, WO1996/040271, WO2010/123983, and U.S. Pat. No. 8,658,608.

In some forms, the triplex-forming molecules can form a triple-stranded molecule with the sequence including GAAAGAAAGAGA (SEQ ID NO:1) or TGCCCTGAAAGAAAGAGA (SEQ ID NO:2) or GGAGAAA (SEQ ID NO:3) or AGAATGGTGCAAAGAGG (SEQ ID NO:4) or AAAAGGG (SEQ ID NO:5) or ACATGATTAGCAAAAGGG (SEQ ID NO:6).

Accordingly, in some forms, the triplex-forming molecule includes the nucleic acid sequence CTTTCTTTCTCT (SEQ ID NO:7), preferably includes the sequence CTTTCTTTCTCT (SEQ ID NO:7) linked to the sequence TCTCTTTCTTTC (SEQ ID NO:8), or more preferably includes the sequence CTTTCTTTCTCT (SEQ ID NO:7) linked to the sequence TCTCTTTCTTTCAGGGCA (SEQ ID NO:9).

In some forms, the triplex-forming molecule includes the nucleic acid sequence TTTCCC (SEQ ID NO:10), preferably includes the sequence TTTCCC (SEQ ID NO:10) linked to the sequence CCCTTTT (SEQ ID NO:11), or more preferably includes the sequence TTTCCC (SEQ ID NO:24) linked to the sequence CCCTTTTGCTAATCATGT (SEQ ID NO:12).

In some forms, the triplex-forming molecule includes the nucleic acid sequence TTTCTCC (SEQ ID NO:13), preferably includes the sequence TTTCTCC (SEQ ID NO:13) linked to the sequence CCTCTTT (SEQ ID NO:14), or more preferably includes the sequence TTTCTCC (SEQ ID NO:13) linked to the sequence CCTCTTTGCACCATTCT (SEQ ID NO:15).

In some forms, the triplex forming nucleic acid is a peptide nucleic acid including the sequence JTTTJTTTJTJT (SEQ ID NO:16) linked to the sequence TCTCTTTCTTTC (SEQ ID NO:8) or TCTCTTTCTTTCAGGGCA (SEQ ID NO:9); or

a peptide nucleic acid including the sequence TTTTJJJ (SEQ ID NO:17) linked to the sequence CCCTTTT (SEQ ID NO:11) or CCCTTTTGCTAATCATGT (SEQ ID NO:12);

or a peptide nucleic acid including the sequence TTTJTJJ (SEQ ID NO:18) linked to the sequence CCTCTTT (SEQ ID NO:14) or

CCTCTTTGCACCATTCT (SEQ ID NO:15),

optionally, but preferably wherein one or more of the PNA residues is a γPNA.

In some forms, the triplex forming molecule is a peptide nucleic acid including the sequence lys-lys-lys-JTTTJTTTJTJT-OOO-T [symbol] T [symbol] T [symbol] T [symbol] T [symbol] T [symbol] A [symbol] G [symbol] C [symbol] -lys-lys-lys (SEQ ID NO:19), or

lys-lys-lys-TTTTJJJ-OOO-C [symbol] C [symbol] T [symbol] T [symbol] C [symbol] A [symbol] T [symbol] A [symbol] G [symbol] -lys-lys-lys (SEQ ID NO:20), or

lys-lys-lys-TTTJTJJ-OOO-C [symbol] T [symbol] T [symbol] T [symbol] C [symbol] C [symbol] A [symbol] T [symbol] T-lys-lys-lys (SEQ ID NO:21);

optionally, but preferably wherein one or more of the PNA residues is a γPNA. In some forms, the bolded and underlined residues are miniPEG-containing γPNA.

In other forms, the triplex forming nucleic acid is a peptide nucleic acid including the sequence TJTTTTJTTJ (SEQ ID NO:22) linked to the sequence CTTCTTTTCT (SEQ ID NO:23); or

TTJTTJTTTJ (SEQ ID NO:24) linked to the sequence CTTTCTTCTT (SEQ ID NO:25); or

JJJTJJTTJT (SEQ ID NO:26) linked to the sequence TCTTCCTCCC (SEQ ID NO:27); or

optionally, but preferably wherein one or more of the PNA residues is a γPNA.

In some forms, the triplex forming nucleic acid is a peptide nucleic acid including the sequence lys-lys-lys-TJTTTTJTTJ-OOO-C [symbol] T [symbol] T [symbol] T [symbol] C [symbol] -lys-lys-lys (SEQ ID NO:42) (IVS2-24); or

lys-lys-lys-TTJTTJTTTJ-OOO-C [symbol] T [symbol] C [symbol] T [symbol] T [symbol] -lys-lys-lys (SEQ ID NO:28) (IVS2-512); or

lys-lys-lys-JJJTJJTTJT-OOO-T [symbol] T [symbol] C [symbol] T [symbol] C [symbol] -lys-lys-lys (SEQ ID NO:29) (IVS2-830);

optionally, but preferably wherein one or more of the PNA residues is a γPNA. In some forms, the bolded and underlined residues are miniPEG-containing γPNA.

2. Sickle Cell Disease

In some forms, the triplex-forming molecule includes the nucleic acid sequence CCTCTTC (SEQ ID NO:30), preferably includes the sequence CCTCTTC (SEQ ID NO:30) linked to the sequence CTTCTCC (SEQ ID NO:31), or more preferably includes the sequence CCTCTTC (SEQ ID NO:30) linked to the sequence CTTCTCCAAAGGAGT (SEQ ID NO:32) or CTTCTCCACAGGAGTCAG (SEQ ID NO:33) or CTTCTCCACAGGAGTCAGGTGC (SEQ ID NO:34).

In some forms, the triplex-forming molecule includes the nucleic acid sequence TTCCTCT (SEQ ID NO:35), preferably includes the sequence TTCCTCT (SEQ ID NO:35) linked to the sequence TCTCCTT (SEQ ID NO:36), or more preferably includes the sequence TTCCTCT (SEQ ID NO:35) linked to the sequence TCTCCTTAAACCTGT (SEQ ID NO:37) or TCTCCTTAAACCTGTCTT (SEQ ID NO:38).

In some forms, the triplex-forming molecule includes the nucleic acid sequence TCTCTTCT (SEQ ID NO:39), preferably includes the sequence TCTCTTCT (SEQ ID NO:39) linked to the sequence TCTTCTCT (SEQ ID NO:40), or more preferably includes the sequence TCTCTTCT (SEQ ID NO:39) linked to the sequence TCTTCTCTGTCTCCAC (SEQ ID NO:41) or TCTTCTCTGTCTCCACAT (SEQ ID NO:79).

In some forms for correction of Sickle Cell Disease Mutation, the triplex forming nucleic acid is a peptide nucleic acid including the sequence JJTJTTJ (SEQ ID NO:43) linked to the sequence CTTCTCC (SEQ ID NO:31) or CTTCTCCAAAGGAGT (SEQ ID NO:32) or CTTCTCCACAGGAGTCAG (SEQ ID NO:33) or CTTCTCCACAGGAGTCAGGTGC (SEQ ID NO:34);

or a peptide nucleic acid including the sequence TTJJTJT (SEQ ID NO:35) linked to the sequence TCTCCTT (SEQ ID NO:36) or TCTCCTTAAACCTGT (SEQ ID NO:37) or TCTCCTTAAACCTGTCTT (SEQ ID NO:38);

or a peptide nucleic acid including the sequence TJTJTTJT (SEQ ID NO:39) linked to the sequence TCTTCTCT (SEQ ID NO:40) or TCTTCTCTGTCTCCAC (SEQ ID NO:41) or TCTTCTCTGTCTCCACAT (SEQ ID NO:79);

optionally, but preferably wherein one or more of the PNA residues is a γPNA.

In forms for correction of Sickle Cell Disease Mutation, the triplex forming nucleic acid is a peptide nucleic acid including the sequence lys-lys-lys-JJTJTTJ-OOO-C [symbol] T [symbol] T [symbol] C [symbol] A [symbol] G [symbol] A [symbol] T-lys-lys-lys (SEQ ID NO:44); or

lys-lys-lys-TTJJTJT-OOO-T [symbol] T [symbol] C [symbol] T [symbol] A [symbol] C [symbol] T [symbol] T-lys-lys-lys (SEQ ID NO:45); or

lys-lys-lys-TTJJTJT-OOO-T [symbol] T [symbol] C [symbol] T [symbol] A [symbol] C [symbol] T [symbol] T [symbol] T [symbol] -lys-lys-lys (SEQ ID NO:46)

lys-lys-lys-TJTJTTJT-OOO-T [symbol] T [symbol] C [symbol] C [symbol] G [symbol] C [symbol] C [symbol] A [symbol] -lys-lys-lys (SEQ ID NO:47) (tc816); or

lys-lys-lys-JJTJTTJ-OOO- [symbol] [symbol] TCTC [symbol] [symbol] C [symbol] G [symbol] A [symbol] T [symbol] A [symbol] -lys-lys-lys (SEQ ID NO:48); or

lys-lys-lys-JJTJTTJ-OOO- [symbol] T [symbol] C [symbol] C [symbol] A [symbol] [symbol] G [symbol] G [symbol] C [symbol] G-lys-lys-lys (SEQ ID NO:48) (SCD-tcPNA 1A); or

lys-lys-lys-JJTJTTJ-OOO- [symbol] -lys-lys-lys (SEQ ID NO:48) (SCD-tcPNA 1B); or

lys-lys-lys-JJ [symbol] J [symbol] J-OOO- [symbol] -lys-lys-lys (SEQ ID NO:48) (SCD-tcPNA 1C); or

lys-lys-lys-JJTJTTJ-OOO- [symbol] T [symbol] C [symbol] C [symbol] A [symbol] A [symbol] G [symbol] G [symbol] C [symbol] G [symbol] TGC-lys-lys-lys (SEQ ID NO:49) (SCD-tcPNA 1D); or

lys-lys-lys-JJTJTTJ-OOO- [symbol] -lys-lys-lys (SEQ ID NO:49) (SCD-tcPNA 1E); or

lys-lys-lys-JJ [symbol] J [symbol] J-OOO- [symbol] -lys-lys-lys (SEQ ID NO:49) (SCD-tcPNA 1F); or

lys-lys-lys-TJTJTTJT-OOO-T [symbol] T [symbol] C [symbol] C [symbol] G [symbol] C [symbol] C [symbol] A [symbol] A [symbol] -lys-lys-lys (SEQ ID NO:60);

optionally, but preferably wherein one or more of the PNA residues is a γPNA. In some forms, the bolded and underlined residues are miniPEG-containing γPNA.

ii. Exemplary Donor Oligonucleotides for Sickle Cell Disease or Beta Thalassemia

In some forms, the triplex forming molecules are used in combination with a donor oligonucleotide for correction of IVS2-654 mutation in thalassemia that includes the sequence 5′AAAGAATAACAGTGATAATTTCTGGGTTAAGGCAATAGCAATA TCTCTGCATATAAATAT 3′ (SEQ ID NO:51) with the correcting IVS2-654 nucleotide underlined, or a functional fragment thereof that is suitable and sufficient to correct the IVS2-654 mutation.

Other exemplary donor sequences include, but are not limited to, DonorGFP-IVS2-1 (Sense) 5′-GTTCAGCGTGTCCGGCGAGGGCG AGGTGAGTCTATGGGACCCTTGATGTTT-3′ (SEQ ID NO:52), DonorGFP-IVS2-1 (Antisense) 5′-AAACATCAAGGGTCCCATA GACTCACCTCGCCCTCGCCGGACACGCTGAAC-3′ (SEQ ID NO:53), and, or a functional fragment thereof that is suitable and sufficient to correct a mutation.

In some forms, a Sickle Cells Disease mutation can be corrected using a donor having the sequence

5′CTTGCCCCACAGGGCAGTAACGGCAGATTTTTC [symbol] CGG CGTTAAATGCACCATGGTGTCTGTTTGAGGT 3′ (SEQ ID NO:54), or a functional fragment thereof that is suitable and sufficient to correct a mutation, wherein the three boxed nucleotides represent the corrected codon 6 which reverts the mutant Valine (associated with human sickle cell disease) back to the wildtype Glutamic acid and nucleotides in bold font (without underlining) represent changes to the genomic DNA but not to the encoded amino acid; or

5′ACAGACACCATGGTGCACCTGACTCCTG [symbol] GGAGAAGTCT GCCGTTACTGCC 3′ (SEQ ID NO:55), or a functional fragment thereof that is suitable and sufficient to correct a mutation, wherein the bolded and underlined residue is the correction, or

5′T(s)T(s)G(s)CCCCACAGGGCAGTAACGGCAGACTTCTCCTC AGG [symbol] GTCAGGTGCACCATGGTGTCTGTT(s)T(s)G(s)3′ (SEQ ID NO:56), or a functional fragment thereof that is suitable and sufficient to correct a mutation, wherein the bolded and underlined residue is the correction and “(s)” indicates an optional phosphorothioate internucleoside linkage.

B. Lung Disorders

i. Triplex Forming Sequences and Donors

1. Cystic Fibrosis

The compositions and methods can be used to treat cystic fibrosis. Cystic fibrosis (CF) is a lethal autosomal recessive disease caused by defects in the cystic fibrosis transmembrane conductance regulator (CFTR), an ion channel that mediates Cl-transport. The most common mutation in CF is a three base-pair deletion (F508del) resulting in the loss of a phenylalanine residue, causing intracellular degradation of the CFTR protein and lack of cell surface expression (Davis, et al., Am J Respir Crit Care Med., 173(5):475-82 (2006)). Of the nonsense mutations G542X and W1282X are the most common with frequencies of 2.6% and 1.6% respectfully.

It has been discovered that triplex-forming PNA molecules and donor DNA can be used to correct mutations leading to cystic fibrosis. In some forms, the compositions are administered by intranasal or pulmonary delivery. In some forms, the triplex-forming molecules can be administered in utero; for example by amniotic sac injection and/or injection into the vitelline vein. The compositions can be administered in an effective amount to induce or enhance gene correction in an amount effective to reduce one or more symptoms of cystic fibrosis.

Sequences for the human CFTR are known in the art, see, for example, GenBank Accession number: AH006034.1, and compositions and methods of targeted correction of CFTR are described in McNeer, et al., Nature Communications, 6:6952, (DOI 10.1038/ncomms7952), 11 pages.

a. Exemplary F508del Target Sites

In some forms, the triplex-forming molecules are designed to target the CFTR gene at nucleotides 9,152-9,159 (TTTCCTCT (SEQ ID NO:57)) or 9,159-9,168 (TTTCCTCTATGGGTAAG (SEQ ID NO:58) of accession number AH006034.1, or the non-coding strand (such as, 3′-5′ complementary sequence) corresponding to nucleotides 9,152-9,159 or 9,152-9,168 (such as, 5′-AGAGGAAA-3′ (SEQ ID NO:59), or 5′-CTTACCCATAGAGGAAA-3′ (SEQ ID NO:50)).

In some forms, the triplex-forming molecules are designed to target the CFTR gene at nucleotides 9,039-9,046 (5′-AGAAGAGG-3′ (SEQ ID NO:61), or 9,030-9,046 (5′-ATGCCAACTAGAAGAGG-3′ (SEQ ID NO:62)) of accession number AH006034.1, or the non-coding strand (such as, 3′-5′ complementary sequence) corresponding to nucleotides (5′ CCTCTTCT 3′ (SEQ ID NO:63)) or (5′ CCTCTTCTAGTTGGCAT 3′ (SEQ ID NO:64).

In some forms, the triplex-forming molecules are designed to target the CFTR gene at nucleotides 8,665-8,683 (CTTTCCCTT (SEQ ID NO:65)) or 8,665-8,682 (CTTTCCCTTGTATCTTTT (SEQ ID NO:66) of accession number AH006034.1, or the non-coding strand (such as, 3′-5′ complementary sequence) corresponding to nucleotides 8,665-8,683 or 8,665-8,682 (such as, 5′-AAGGGAAAG-3′ (SEQ ID NO:67), or 5′-AAAAGATAC AAGGGAAAG-3′ (SEQ ID NO:68)).

In some forms, the triplex-forming molecules are designed to target the W1282X mutation in CFTR gene at the sequence GAAGGAGAAA (SEQ ID NO:69), AAAAGGAA (SEQ ID NO:70), or AGAAAAAAGG (SEQ ID NO:71), or the inverse complement thereof.

In some forms, the triplex-forming molecules are designed to target the G542X mutation in CFTR gene at the sequence AGAAAAA (SEQ ID NO:72), AGAGAAAGA (SEQ ID NO:73), or AAAGAAA (SEQ ID NO:74), or the inverse complement thereof.

b. Exemplary Triplex Forming Sequences and Donors F508del

In some forms, the triplex-forming molecule includes the nucleic acid sequence includes TCTCCTTT (SEQ ID NO:75), preferably linked to the sequence TTTCCTCT (SEQ ID NO:57) or more preferably includes TCTCCTTT (SEQ ID NO:75) linked to the sequence TTTCCTCTATGGGTAAG (SEQ ID NO:58); or

includes TCTTCTCC (SEQ ID NO:78) preferably linked to the sequence CCTCTTCT (SEQ ID NO:63), or more preferably includes TCTTCTCC (SEQ ID NO:78) linked to CCTCTTCTAGTTGGCAT (SEQ ID NO:64); or

includes TTCCCTTTC (SEQ ID NO:76), preferably includes the sequence TTCCCTTTC (SEQ ID NO:76) linked to the sequence CTTTCCCTT (SEQ ID NO:65), or more preferably includes the sequence TTCCCTTTC (SEQ ID NO:76) linked to the sequence CTTTCCCTTGTATCTTTT (SEQ ID NO:66).

In some forms, the triplex forming nucleic acid is a peptide nucleic acid including the sequence TJTJJTTT (SEQ ID NO:77, linked to the sequence TTTCCTCT (SEQ ID NO:57) or TTTCCTCTATGGGTAAG (SEQ ID NO:58); or

TJTTJTJJ (SEQ ID NO:84) linked to the sequence CCTCTTCT (SEQ ID NO:63), or CCTCTTCTAGTTGGCAT (SEQ ID NO:80); or

TTJJJTTTJ (SEQ ID NO:85) linked to the sequence CTTTCCCTT (SEQ ID NO:65), or CTTTCCCTTGTATCTTTT (SEQ ID NO:66);

optionally, but preferably wherein one or more of the PNA residues is a γPNA.

In some forms the triplex forming nucleic acid is a peptide nucleic acid including the sequence is lys-lys-lys-TJTJJTTT-OOO- [symbol] T [symbol] C [symbol] CTA [symbol] G [symbol] GG [symbol] A [symbol] G-lys-lys-lys (SEQ ID NO:86) (hCFPNA2); or lys-lys-lys- [symbol] J [symbol] JJ [symbol] T [symbol] -OOO-TTTCCTCTATGGGTAAG-lys-lys-lys (SEQ ID NO:86); or

lys-lys-lys-TJTTJTJJ-OOO-C [symbol] T [symbol] T [symbol] CTAGT [symbol] G [symbol] C [symbol] T-lys-lys-lys (SEQ ID NO:87) (hCFPNA1); or

lys-lys-lys-TJJJTTTJ-OOO-C [symbol] T [symbol] C [symbol] C [symbol] T [symbol] T [symbol] T [symbol] T [symbol] T [symbol] -lys-lys-lys (SEQ ID NO:88) (hCFPNA3);

optionally, but preferably wherein one or more of the PNA residues is a γPNA. In some forms, the bolded and underlined residues are miniPEG-containing γPNA.

In some forms, a donor that can be used for CFTR gene correction, particularly in combination with the foregoing triplex forming molecules, includes the sequence 5′TTCTGTATCTATATTCATCATAGGAAACACCAAAGATAATGTTCT CCTTAATGGTGCCAGG3′ (SEQ ID NO:89), or a functional fragment thereof that is suitable and sufficient to correct the F508del mutation in the cystic fibrosis transmembrane conductance regulator (CFTR) gene.

W1282 Mutation Site

In some forms, the triplex-forming molecule includes the nucleic acid sequence CTTCCTCTTT (SEQ ID NO:90), preferably includes the sequence CTTCCTCTTT (SEQ ID NO:90) linked to the sequence TTTCTCCTTC (SEQ ID NO:91), or more preferably includes the sequence CTTCCTCTTT (SEQ ID NO:90) linked to the sequence TTTCTCCTTCAGTGTTCA (SEQ ID NO:92); or

the triplex-forming molecule includes the nucleic acid sequence TTTTCCT (SEQ ID NO:93), preferably includes the sequence TTTTCCT (SEQ ID NO:93) linked to the sequence TCCTTTT (SEQ ID NO:94), or more preferably includes the sequence TTTTCCT (SEQ ID NO:93) linked to the sequence TCCTTTTGCTCACCTGTGGT (SEQ ID NO:95); or

the triplex-forming molecule includes the nucleic acid sequence TCTTTTTTCC (SEQ ID NO:96), preferably includes the sequence TCTTTTTTCC (SEQ ID NO:96) linked to the sequence CCTTTTTTCT (SEQ ID NO:97), or more preferably includes the sequence TCTTTTTTCC (SEQ ID NO:96) linked to the sequence CCTTTTTTCTGGCTAAGT (SEQ ID NO:98).

In some forms, the triple forming nucleic acid is a peptide nucleic acid including the sequence JTTJJTJTTT (SEQ ID NO:99) linked to the sequence TTTCTCCTTC (SEQ ID NO:91) or TTTCTCCTTCAGTGTTCA (SEQ ID NO:92); or

a peptide nucleic acid including the sequence TTTTJJT (SEQ ID NO:100) linked to the sequence TCCTTTT (SEQ ID NO:94) or linked to the sequence TCCTTTTGCTCACCTGTGGT (SEQ ID NO:95); or

a peptide nucleic acid including the sequence TJTTTTTTJJ (SEQ ID NO:101) linked to the sequence CCTTTTTTCT (SEQ ID NO:97) or linked to the sequence CCTTTTTTCTGGCTAAGT (SEQ ID NO:98);

optionally, but preferably wherein one or more of the PNA residues is a γPNA.

In some forms, the triplex forming nucleic acid is a peptide nucleic acid including the sequence lys-lys-lys-JTTJJTJTTT-OOO-T [symbol] T [symbol] T [symbol] C [symbol] T [symbol] A [symbol] TGT [symbol] C [symbol] -lys-lys-lys (SEQ ID NO:102) (tcPNA-1236); or

lys-lys-lys-TTTTJJT-OOO-T [symbol] C [symbol] T [symbol] T [symbol] C [symbol] C [symbol] C [symbol] T [symbol] T [symbol] G [symbol] -lys-lys-lys (SEQ ID NO:103) (tcPNA-1314); or

lys-lys-lys-TJTTTTTTJJ-OOO-C [symbol] T [symbol] T [symbol] T [symbol] C [symbol] G [symbol] C [symbol] A [symbol] G [symbol] -lys-lys-lys (SEQ ID NO:104) (tcPNA-1329);

optionally, but preferably wherein one or more of the PNA residues is a γPNA. In some forms, the bolded and underlined residues are miniPEG-containing γPNA.

In some forms, a donor that can be used for CFTR gene correction, particularly in combination with the foregoing triplex forming molecules, includes the sequence T(s)C(s)T(s)-TGGGATTCAATAAC [symbol] TTGCA [symbol] ACAGTG [symbol] AGGAA [symbol] GCCTTTGG [symbol] G TGATACCACAGG-(s)T(s)G(s) (SEQ ID NO:105) or a functional fragment thereof that is suitable and sufficient to correct a mutation in CFTR, wherein the bolded and underlined nucleotides are inserted mutations for gene correction, and “(s)” indicates an optional phosphorothioate internucleoside linkage.

G542X Mutation Site

In some forms, the triplex-forming molecule includes the nucleic acid sequence TCTTTTT (SEQ ID NO:106), preferably includes the sequence TCTTTTT (SEQ ID NO:106) linked to the sequence TTTTTCT (SEQ ID NO:107), or more preferably includes the sequence TCTTTTT (SEQ ID NO:106) linked to the sequence TTTTTCTGTAATTTTTAA (SEQ ID NO:108); or

the triplex-forming molecule includes the nucleic acid sequence TCTCTTTCT (SEQ ID NO:109), preferably includes the sequence TCTCTTTCT (SEQ ID NO:109) linked to the sequence TCTTTCTCT (SEQ ID NO:110), or more preferably includes the sequence TCTCTTTCT (SEQ ID NO:109) linked to the sequence TCTTTCTCTGCAAACTT (SEQ ID NO:111); or

the triplex-forming molecule includes the nucleic acid sequence TTTCTTT (SEQ ID NO:112), preferably includes the sequence TTTCTTT (SEQ ID NO:112) linked to the sequence TTTCTTT (SEQ ID NO:112), or more preferably includes the sequence TTTCTTT (SEQ ID NO:112) linked to the sequence TTTCTTTAAGAACGAGCA (SEQ ID NO:113).

In some forms, the triple forming nucleic acid is a peptide nucleic acid including the sequence TJTTTTT (SEQ ID NO:114) linked to the sequence TTTTTCT (SEQ ID NO:107) or TTTTTCTGTAATTTTTAA (SEQ ID NO:108); or

a peptide nucleic acid including the sequence TJTJTTTJT (SEQ ID NO:115) linked to the sequence TCTTTCTCT (SEQ ID NO:110) or linked to the sequence TCTTTCTCTGCAAACTT (SEQ ID NO:111); or

a peptide nucleic acid including the sequence TTTJTTT (SEQ ID NO:116) linked to the sequence TTTCTTT (SEQ ID NO:112) or linked to the sequence TTTCTTTAAGAACGAGCA (SEQ ID NO:113);

optionally, but preferably wherein one or more of the PNA residues is a γPNA.

In some forms, the triplex forming nucleic acid is a peptide nucleic acid including the sequence lys-lys-lys-TJTTTTT-OOO-T [symbol] T [symbol] T [symbol] T [symbol] T [symbol] A [symbol] T [symbol] T [symbol] A [symbol] -lys-lys-lys (SEQ ID NO:80) (tcPNA-302); or

lys-lys-lys-TJTJTTTJT-OOO-T [symbol] T [symbol] T [symbol] T [symbol] T [symbol] C [symbol] A [symbol] C [symbol] T-lys-lys-lys (SEQ ID NO:81) (tcPNA-529); or

lys-lys-lys-TTTJTTT-OOO-T [symbol] T [symbol] T [symbol] T [symbol] A [symbol] A [symbol] C [symbol] A [symbol] C [symbol] -lys-lys-lys (SEQ ID NO:82) (tcPNA-586);

optionally, but preferably wherein one or more of the PNA residues is a γPNA. In some forms, the bolded and underlined residues are miniPEG-containing γPNA.

In some forms, a donor that can be used for CFTR gene correction, particularly in combination with the foregoing triplex forming molecules, includes the sequence T(s)C(s)C(s)-AAGTTTGCAGAGAAAGA [symbol] AATATAGT [symbol] CTT [symbol] GAGAAGG [symbol] GGAAT CAC [symbol] CTGAGTGGA-G(s)G(s)T(s) (SEQ ID NO:83), or a functional fragment thereof that is suitable and sufficient to correct a mutation in CFTR, wherein the bolded and underlined nucleotides are inserted mutations for gene correction, and “(s)” indicates an optional phosphorothioate internucleoside linkage.

The formulations can be administered in a single dose or in multiple doses. Certain factors may influence the dosage required to effectively treat a subject, including but not limited to the severity of the disease or disorder, previous treatments, the general health and/or age of the subject, and other diseases present. It will also be appreciated that the effective dosage of the formulation used for treatment may increase or decrease over the course of a particular treatment. Changes in dosage may result and become apparent from the results of diagnostic assays.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the subject. Persons of ordinary skill can easily determine optimum dosages, dosing methodologies and repetition rates. Optimum dosages may vary depending on the relative potency of individual agents, and can generally be estimated based on EC₅₀ values found to be effective in vitro and in vivo animal models.

Those skilled in the art will recognize, or be able to ascertain using no more than routine experimentation, many equivalents to the specific embodiments of the invention described herein. Such equivalents are intended to be encompassed by the following claims.

### EXAMPLES

NPs are useful for drug delivery because of their potential to target specific tissues (Patra, et al., Journal of Nanobiotechnology 2018, 16(1): 71). The effect of PLGA NP size on in vivo biodistribution after intravenous administration was explored. Microfluidic systems (Kucuk and Edirisinghe, Journal of Nanoparticle Research: An Interdisciplinary Forum For Nanoscale Science and Technology 2014, 16(12): 2626-2626) were used for fine-tuning of NP size. One such microfluidic system is the benchtop NanoAssemblr™ from Precision NanoSystems Inc. Here, the NanoAssemblr™ was used to produce PLGA NPs of controlled size (Morikawa, et al., Biological and Pharmaceutical Bulletin 2018, 41(6): 899-907). Two methods were used to quantify the effects of PLGA NP size in bulk tissue following systemic administration: an in vivo imaging system (IVIS) and flow cytometry. Nanoparticle internalization and accumulation in cells were visualized. It was shown that after intravenous administration, NPs approximately 120 nm in diameter access lung and bone marrow compartments in greater numbers than NPs of 160 nm in diameter or larger. This sharp threshold for size-dependent accumulation provides important guidance to the design of nanomaterials for drug delivery to the bone marrow and lung.

### Example 1: Biodegradable NP Size for Tissue- and/or Cell-Selective Uptake

### Materials and Methods

(i) Materials

Poly(D,L-lactide-co-glycolide; Mn=10-15 kDa, LA:GA=50:50) was purchased from PolySciTech (West Lafayette, Ind.). 1,1′-dioctadecyl-3,3,3′,3′tetramethylindodicarbocyanine, 4-chlorobenzenesulfonate salt (DiD) was ordered from Biotium (Fremont, Calif.). Acetonitrile (ACN) and dimethyl sulfoxide (DMSO) were obtained from J.T. Baker (Phillipsburg, N.J.). 30-50 kDa poly(vinyl alcohol) (PVA) and Bovine Serum Albumin (BSA) were obtained from Sigma-Aldrich (St. Louis, Mo.). Amicon Ultra-15 filter tubes and heparin were obtained from MilliporeSigma (Burlington, Mass.). 378.3 g mol⁻¹ D-(+)-trehalose dehydrate (trehalose) was purchased from MP Biomedicals (Irvine, Calif.). Slide-A-Lyzer MINI Dialysis Devices with 10K MWCO, Hoescht 33342, and CellTrace™ CFSE were purchased from Thermo Fisher (Waltham, Mass.). Tissue-Tek O.C.T. Compound was obtained from Sakura Finetek (Torrance, Calif.). DAKA Fluorescence Mounting Medium was purchased from Agilent Technologies (Santa Clara, Calif.). Chamber slides were purchased from Lab-Tek (Grand Rapids, Mich.). IsoThesia (Isoflurane) solution was obtained from Henry Schein Animal Health (Dublin, Ohio). 40 μm and 70 μm Sterile Cell Strainers were purchased from Fisher Scientific (Hampton, N.H.). RPMI 1640 Medium was purchased from Gibco (Gaithersberg, Md.). Fetal Bovine Serum (FBS) was purchased from Atlanta Biologicals (Flowery Branch, Ga.). Collagenase, Type 2 was purchased from Worthington (Lakewood, N.J.). ACK Lysing Buffer contained 154.95 mM ammonium chloride from JT Baker, 10 mM potassium bicarbonate from Sigma Aldrich, and 0.1 mM EDTA from Thermofisher. LIVE/DEAD Fixable Green Dead Cell Stain was ordered from Invitrogen. Antibody stains anti-mouse P2X7R, anti-mouse CD31, and anti-mouse F480 were purchased from BioLegend (San Diego, Calif.). EasySep Mouse CD117 (cKIT) Positive Selection Kit was obtained from StemCell Technologies (Vancouver, Canada).

(ii) Fabrication of NPs

PLGA NPs were produced using a benchtop NanoAssemblr™ instrument (Precision NanoSystems Inc., Vancouver, Canada). NPs with a range of sizes were formulated by dissolving PLGA at various concentrations in ACN. Fifteen mg of polymer was dissolved in 3 mL of ACN overnight to achieve NP-1, 20 mg of polymer was dissolved in 1 mL of ACN overnight to achieve NP-2, 30 mg of polymer was dissolved in 1 mL of ACN overnight to achieve NP-3, and 40 mg of polymer was dissolved in 1 mL of ACN overnight to achieve NP-4. To trace NPs in tissues, NPs were loaded with 0.5% DiD, a hydrophobic dye that has been used as a marker for NPs in prior studies (Deng, et al., Biomaterials 2014, 35(24): 6595-6602; Hu, et al., Proceedings of the National Academy of Sciences 2011, 108(27): 10980). For dye-loaded NPs, 10 mg of DiD dissolved in 1 mL of dimethyl sulfoxide (DMSO) was added to the polymer solution at 0.5% wt:wt DiD:PLGA.

The organic phase containing the polymer/dye solution was injected into one port of the NanoAssemblr™ instrument. The aqueous phase containing 2% w:v PVA was simultaneously injected into the second port of the system to maintain a 1:1 aqueous:organic flow rate ratio. The total flow rate was maintained at 8 mL min⁻¹. NP product was gathered in a 15 mL Falcon tube containing 2 mL of water, while separately disposing the initial 0.25 mL and the final volume of 0.05 mL of the NP solution. DI water was immediately added to bring the NPs solution to a volume of 15 mL and transferred to an Amicon Ultra-15 filter tube (100 K cutoff). NPs were washed at 4,000 g at 4° C. for 45 min three times with DI water. Subsequently, the NPs were resuspended in 1 mg trehalose per 1 mg of NP, frozen at −80° C. and then lyophilized NPs were stored at −20° C. after lyophilization until use.

(iii) In Vitro Characterization of NPs

The hydrodynamic diameter, polydispersity index (PDI), and surface charge (zeta-potential) of each NP formulation were measured at 0.05 mg mL⁻¹ in water by dynamic light scattering (DLS) using a Zetasizer Nano-ZS (Malvern Instruments). A gold-palladium sputter coating was applied to NPs and morphologies were characterized by scanning electron microscopy (SEM) using a XL-30 scanning electron microscope (FEI/Philips).

To determine the loading of DiD in each NP formulation, 2 mg of NPs were resuspended by vortex and water bath sonication in 100 μL DMSO. Two-fold serial dilutions of the NP solutions were made into water and the concentration of DiD dye was quantified using a plate reader (ex/em 644/665 nm). Dye loading was calculated from a standard curve.

(iv) Animal Preparation

All procedures and experiments were performed in accordance with the guidelines and policies of the Yale Animal Resource Center (YARC) and approved by the Yale University Institutional Animal Care and Use Committee (IACUC). Male C57BL/6 mice (6-8 weeks old) obtained from Charles River Laboratories were used.

(v) IVIS Ex Vivo Biodistribution of NPs in Whole Organs

2 mg of fluorescent NPs were resuspended by vortex and water bath sonication in 1×dPBS to a concentration of 10 mg mL⁻¹ and administered retro-orbitally to mice (n=3). After 24 h, mice were sacrificed and perfused with heparinized saline. Tissues (brain, heart, lungs, liver, kidneys, spleen, pancreas, and bone marrow) were harvested for ex vivo imaging. Tissues were washed briefly in 1×PBS and imaged using a live imaging instrument (IVIS Spectrum, PerkinElmer) (ex/em 644/665 nm).

(vi) Cellular Biodistribution of NPs

Flow cytometry was used to further assess and quantify NP biodistribution in vivo. Two mg of NPs were resuspended by vortex and water bath sonication in 1×dPBS to a concentration of 10 mg mL⁻¹ and administered retro-orbitally to mice (n=3). After 24 h, mice were sacrificed, perfused with heparinized saline, and tissues (brain, heart, lung, liver, kidney, spleen, pancreas, and bone marrow) were harvested. Tissues were homogenized to single cell suspensions in RPMI 1640 Medium through a 70 μm cell strainer. The resulting single cell suspensions were pelleted and resuspended in 200 μL of PBS containing 1% BSA (FACS buffer). The cells were stained with LIVE/DEAD Fixable Green Dead Cell Stain for 30 min at 4° C. After staining, cells were washed once with FACS buffer for 10 min and then resuspended in 200 μL of FACS buffer. Cell fluorescence was quantified using flow cytometry (Attune NxT, Invitrogen).

(vii) NP Uptake in Type I Alveolar Epithelial Cells, Endothelial Cells, and Alveolar Macrophage Cells

Flow cytometry was used to assess NP distribution in specific lung cell populations. Mice (n=3) were dosed with 2 mg of NP-1, NP-2, NP-3, and NP-4 at a concentration of 10 mg mL⁻¹. After 24 hours, mice were sacrificed, perfused with heparinized saline, and lung tissue was harvested. The tissue was minced into several small pieces, incubated in 0.4% collagenase for 40 min at 37° C. while shaking, and then homogenized through a 70 μm cell strainer. The resulting single cell suspensions were pelleted, resuspended in 2 mL of ACK Lysing Buffer, and incubated for 2 min at room temperature to lyse red blood cells and remove debris. To neutralize the ACK Lysing Buffer, 8 mL of RPMI 1640 containing 10% FBS was added to the solution. The cells were pelleted and resuspended in 200 μL of FACS buffer. The cells were stained with LIVE/DEAD Fixable Green Dead Cell Stain for 30 min at 4° C. After staining, cells were washed once with FACS buffer for 10 min and then incubated with anti-CD31 antibody, anti-F480 antibody, and anti-P2X7R antibody for 30 min After staining, cells were washed once with FACS buffer for 10 min and then resuspended in 200 μL of FACS buffer. Cell fluorescence was quantified using flow cytometry.

(viii) NP Uptake in Hematopoietic Stem and Progenitor Cells

Flow cytometry was used to assess NP distribution in bulk bone marrow and hematopoietic stem and progenitor cells. Mice (n=3) were treated with 2 mg of NP-1, NP-2, NP-3, and NP-4 at a concentration of 10 mg mL⁻¹. After 24 h, mice were sacrificed, perfused with heparinized saline, and femur and tibias harvested. Femurs and tibias were flushed with 5 mL 1×PBS (Madaan, et al., Journal of Biological Methods 2014, 1(1): e1; doi: 10.14440/jbm.2014.12). Bone marrow cells were filtered through a 70 μm cell strainer and washed once with 1×PBS for 10 min Hematopoietic stem and progenitor cells (HSPCs) were selected using the EasySep Mouse CD117 (cKIT) Positive Selection Kit (StemCell Technologies). CD117⁺ cells were stained with LIVE/DEAD Fixable Green Dead Cell Stain for 30 min at 4° C. After staining, CD117⁺ cells were washed once with FACS buffer for 10 min and then resuspended in 200 μL FACS buffer. Cell fluorescence was quantified using flow cytometry.

(ix) Release Kinetics of Agent

The release of DiD dye from PLGA NPs was analyzed using established methods (Deng, et al., Biomaterials 2014, 35(24), 6596-6602; Hu, et al., Proc. Natl. Acad. Sci. USA 2011, 108(27), 10980). Briefly, 1 mg of NPs was resuspended by vortex and water bath sonication in 100 μL PBS, and then loaded into a Slide-A-Lyzer MINI Dialysis Device. The NPs were dialyzed in PBS at 37° C. while shaking and the PBS solution was replaced at each pre-determined time point. For each time point, the nanoparticle solutions in the dialysis units were collected and the dye was quantified using a plate reader (ex/em 644/665).

(x) Fluorescence Microscopy and Nanoparticle Foci Analysis

Confocal microscopy was completed with an additional three animals for each treatment group, to measure cellular internalization of nanoparticles. Bone marrow cells were stained using CellTrace™ CFSE according to the manufacture's protocol. 200,000 stained cells per 400 μL were seeded in chamber slides and fixed by adding an equal amount of 4% PFA in PBS to the media followed by a 15 min centrifugation step at 800 RCF in a swing bucket centrifuge. Subsequently, media/PFA mix was replaced with 4% PFA in PBS and samples were centrifuged again at 800 RCF for 15 min DNA was stained with 2 μg/mL Hoechst 33342 in PBS for 15 min at room temperature. Lungs from at least three animals per condition were harvested, frozen in O.C.T. Compound, and sectioned. Ten μm sections were subsequently stained with 2 μg/mL Hoechst 33342 in PBS for 30 min at room temperature. After washing twice with PBS, bone marrow cells and lung samples were covered with coverslips using DAKO Fluorescence Mounting Medium. Images were analyzed with a Nikon Eclipse Ti fluorescence microscope with a Plan Apo 60X/1.40 Oil DIC h objective, a CSU-W1 confocal scanning unit with an iXon Ultra camera (Andor Technology), MLC 400B laser unit (Agilent Technologies), and NIS Elements 4.30 software (Nikon Corporation). Whole cell and nuclear NP foci were analyzed with the Focinator v2-31 software as previously described (Oeck, et al., Scientific Reports 2019, 9(1), 3148-3148). Images of the lung tissue section were quantified with the Stripenator software as previously described (Oeck, et al., Scientific Reports 2019, 9(1), 3148-3148). Representative images were generated using ImageJ.

(xi) Data Analysis

FlowJo v10.5.2 software was used to analyze flow cytometry data. GraphPad Prism 7 software was used for graphing and statistical analysis. Error bars represent standard error of the mean (SEM). Statistical significance was calculated by either a one-way ANOVA with a Bonferroni's multiple comparisons test or an unpaired t-test (α=0.05), which have been designated appropriately in each figure caption. Significance is represented on plots as: not significant, ns, p>0.05; *p≤0.05; **p≤0.01; ***p≤0.001; and ****p≤0.0001.

Results

(i) Formulation and Characterization of NPs

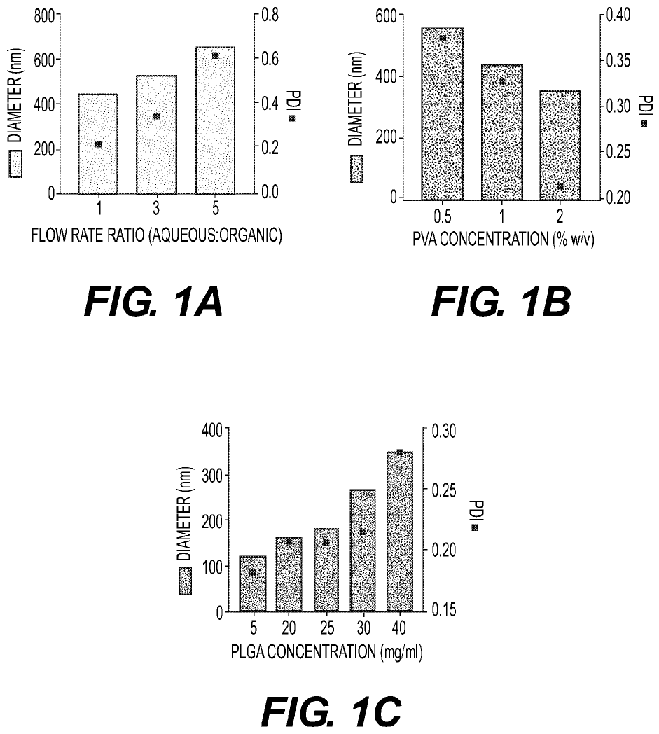

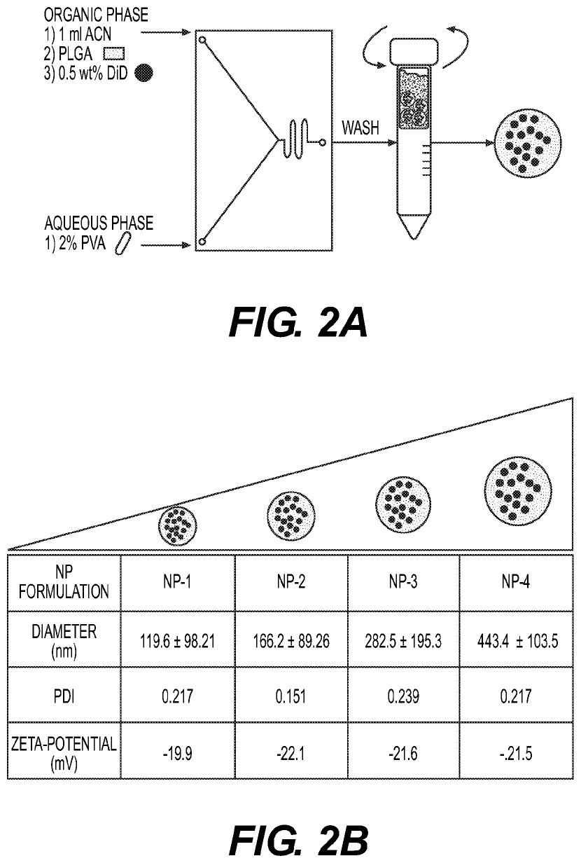

To test whether the NanoAssemblr™ could be used to formulate DiD-loaded PLGA NPs of various sizes, several operational parameters-including flow rate ratio, stabilizer concentration, and PLGA concentration—were systematically varied and the size of resulting NPs was measured. Stable NPs with a hydrodynamic diameter less than 500 nm and PDI less than 0.25 were produced by maintaining an aqueous to organic phase flow rate ratio of 1:1 and surfactant concentration of 2% (FIGS. 1A and 1B). NPs with a range of sizes less than 500 nm were engineered by altering the concentration of PLGA dissolved in ACN (FIG. 1C). Based on these results, experimental conditions were identified for reproducible synthesis of 4 distinct NP populations: NP-1, NP-2, NP-3, and NP-4. The diameter of NP-1 was approximately 120 nm, NP-2 was 160 nm, NP-3 was 280 nm, and NP-4 was 440 nm (FIG. 1B). The PDIs of all NP formulations were less than 0.25 (FIG. 2B). These results were confirmed by SEM, which also demonstrated that NP morphology was spherical and fairly uniform for all NP formulations. All NP formulations had a zeta-potential near −20 mV (FIG. 2B). Further, all NPs demonstrated comparable DiD loading of 9 μg per mg of NP.

The sizes of the nanoparticles (NP-1 and NP-2) were measured before washing, after washing, and after lyophilization to investigate effect(s) of any of these processing steps on the size of the nanoparticles. The results showed that these processing steps did not affect nanoparticle size. Additional analysis of the sizes of the nanoparticles also showed that the nanoparticle sizes were reproducible for separate runs in the microfluidic system.

(ii) IVIS Biodistribution in Whole Organs Depends on NP Size

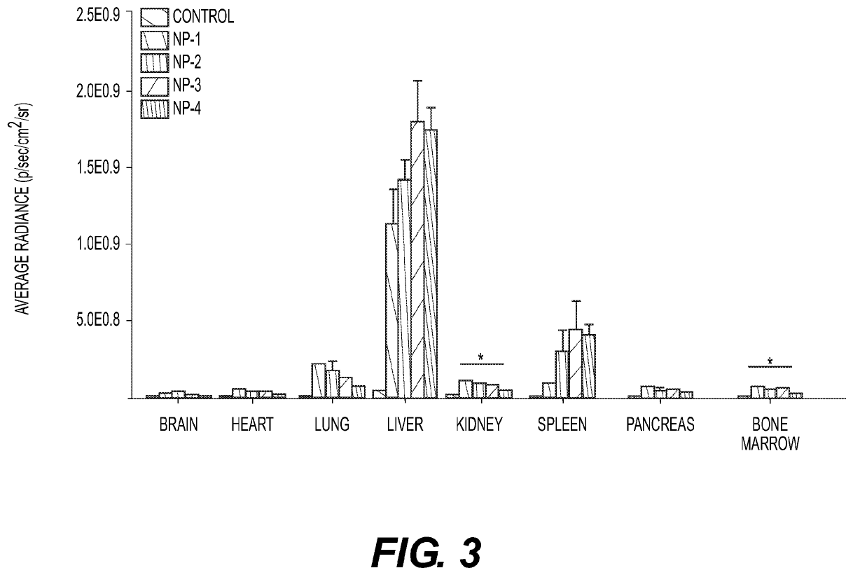

To test whether size of PLGA NPs influences whole tissue accumulation, DiD-loaded NPs were administered intravenously by retro-orbital injection. After 24 h, brain, heart, lung, liver, kidney, spleen, pancreas, and bone marrow tissue were excised and imaged using IVIS. An average radiance (p/sec/cm²/sr) of DiD fluorescence was quantified using a tissue-specific region of interest (FIG. 3). Rigorous inter-organ statistical analyses were conducted to compare NP formulations to the untreated control and NP formulations to each other. The average radiance in the brain and heart tissue was comparable to the untreated control for all NP formulations. In the lung, kidney, pancreas, and bone marrow, NP-1 demonstrated the greatest fluorescence accumulation while NP-4 demonstrated the lowest fluorescence accumulation. Further, the average radiance of NP-1 and NP-2 were significantly enhanced in the lung compared to the untreated control. The average radiance of NP-1, NP-2, and NP-3 were significantly enhanced in the kidney and bone marrow compared to the untreated control. The average radiance of NP-1 was significantly enhanced in the pancreas compared to the untreated control.

Liver tissue showed the highest fluorescence accumulation of all tissues analyzed. While all NP formulations demonstrated significant accumulation in liver, NP-3 and NP4 had the greatest average radiance in the liver. The average radiance in spleen tissue approached 5.0E0.8 p/sec/cm²/sr, with NP-1 having the lowest fluorescence accumulation and NP-3 having the greatest fluorescence accumulation.

(iii) In Vivo Biodistribution in Bulk Tissue Depends on NP Size

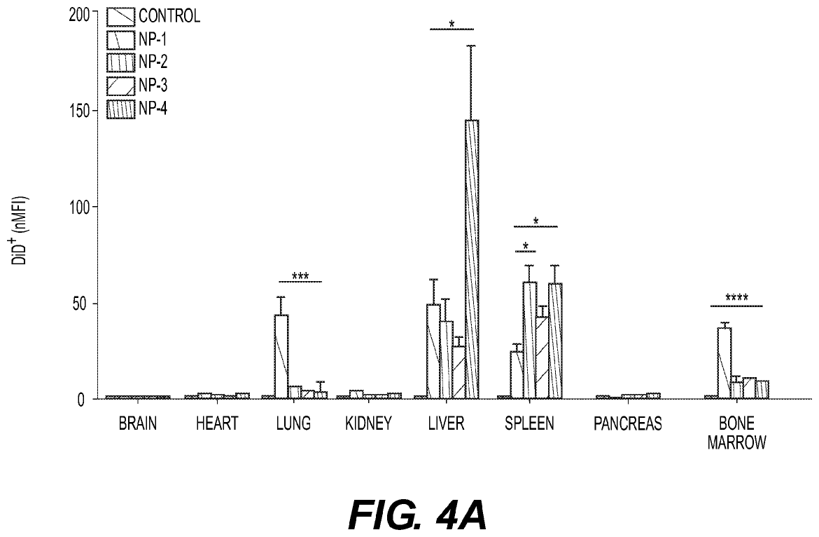

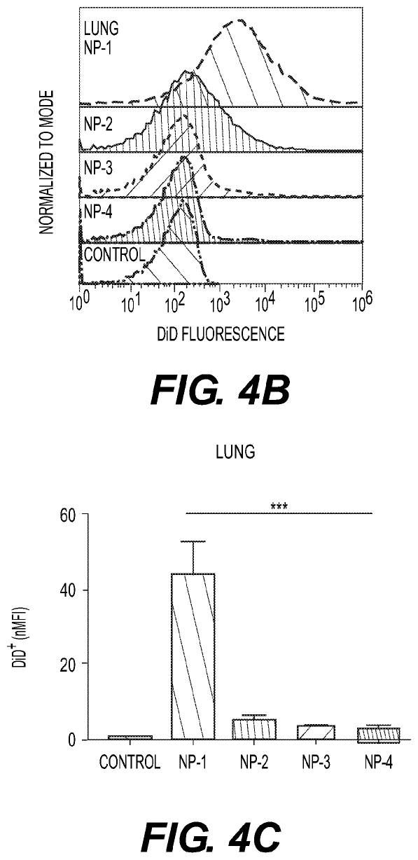

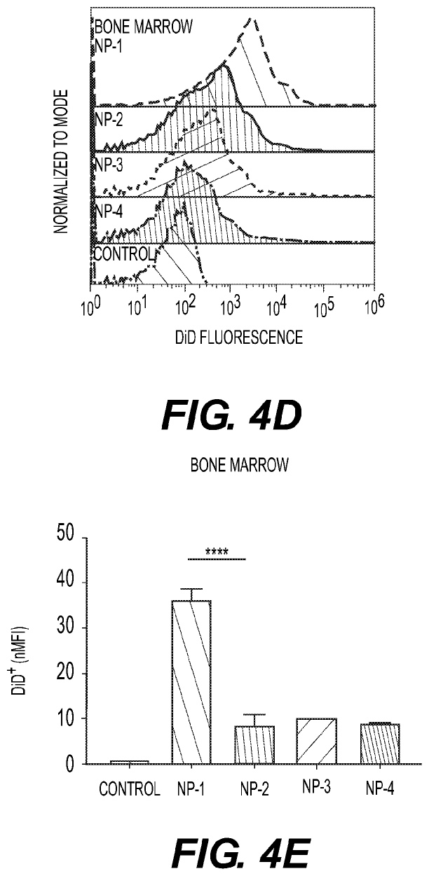

To further understand the effect of NP size on in vivo distribution, flow cytometry was used to assess cellular uptake. Twenty-four h after NP administration, tissues were excised and homogenized to form single cell suspensions. The mean fluorescence intensity of each NP formulation was quantified in all cells and normalized to the mean fluorescence intensity of the untreated control (nMFI) (FIG. 4A). Rigorous inter-organ statistical analyses were conducted to compare NP formulations to the untreated control and NP formulations to one another. The nMFI in brain, heart, and pancreas tissue after injection of all NP formulations was comparable to the untreated control. In the kidney, NP-1 demonstrated significantly enhanced uptake in comparison to the untreated control. The majority of the NPs accumulated in lung, liver, spleen, and bone marrow. In the liver, NP-4 had a significantly enhanced nMFI compared with the untreated control and all other NP formulations. In the spleen, injection of NP-1, NP-2, NP-3, and NP-4 formulations led to a significantly greater nMFI compared to the untreated control. In the lung, NP-1 had a significantly greater nMFI compared with the untreated control and all other NP formulations (FIGS. 4B and 4C). Similarly, in bone marrow, NP-1 had a significantly greater nMFI compared with all the untreated control and all other NP formulations (FIGS. 4D and 4E).

(iv) Flow Cytometry Reveals Enhanced Uptake of NP-1 in Type I Alveolar Epithelial and Alveolar Macrophage Cells

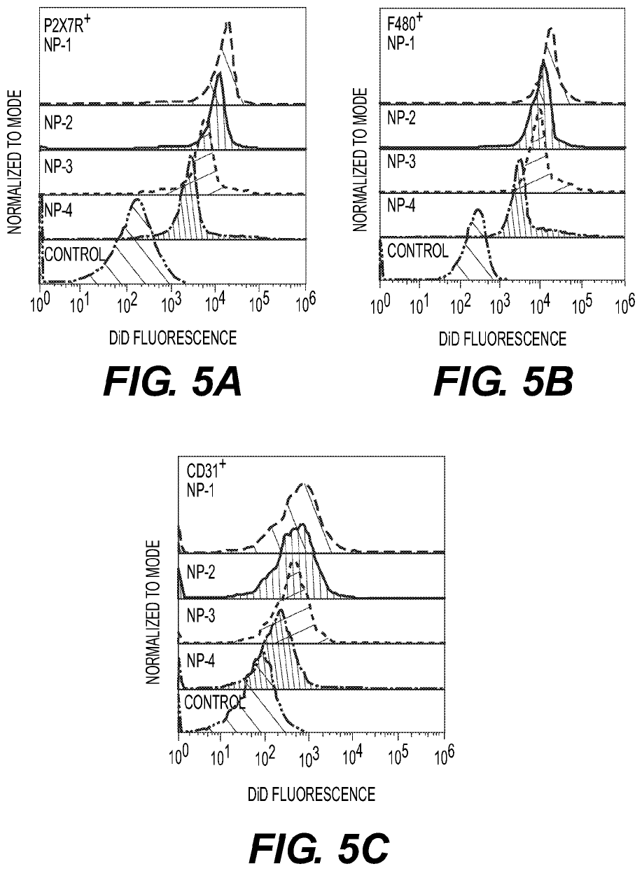

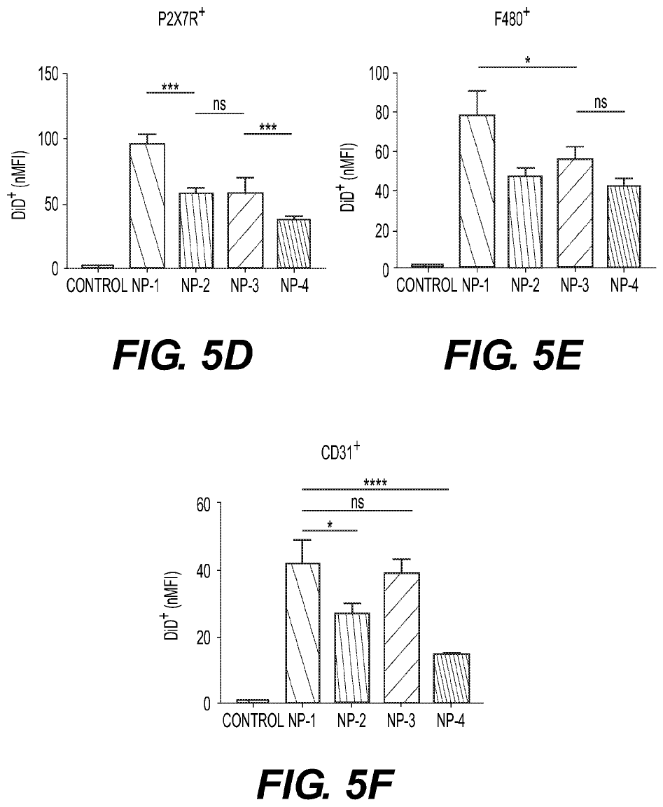

Since NP-1 exhibited extensive uptake in lung tissue, the specific lung cell populations in which NPs accumulated were investigated. Twenty-four h after injection, lung tissue was harvested, digested, and processed into a single cell suspension for flow cytometry. Type I alveolar epithelial cells (AEC I) were identified by staining with antibodies to P2X7R⁺ and were found to represent 14.4% of the overall lung cell population. Alveolar macrophages were identified by staining with antibodies to F480⁺ and were found to represent 17.7% of the overall lung cell population. Endothelial cells were identified by staining with antibodies to CD31⁺ and were found to represent 13.3% of the overall lung cell population. At 24 h, NP-1, NP-2, NP-3, and NP-4 treated mice resulted in an increase in DiD fluorescence in AEC I (FIG. 5A), alveolar macrophages (FIG. 5B), and endothelial cells (FIG. 5C) compared to the untreated controls. The nMFI for NP-1 treated mice was significantly greater than NP-2, NP-3, and NP-4 treated mice in AEC I (FIG. 5D). In AEC I, the nMFI was not significantly different between NP-2 and NP-3 treated mice, however the nMFI was significantly different between NP-2 and NP-4 treated mice. A significant increase in nMFI was observed for NP-1 treated mice in comparison with all other NP formulations in alveolar macrophages (FIG. 5E). NP-2, NP-3, and NP-4 treated mice had comparable nMFI values in alveolar macrophages. In endothelial cells, NP-1 and NP3 treated mice did not have significantly different nMFIs, however both NP-1 and NP-3 presented a significant increase in nMFI over NP-2 and NP-4 treated and control mice (FIG. 5F).

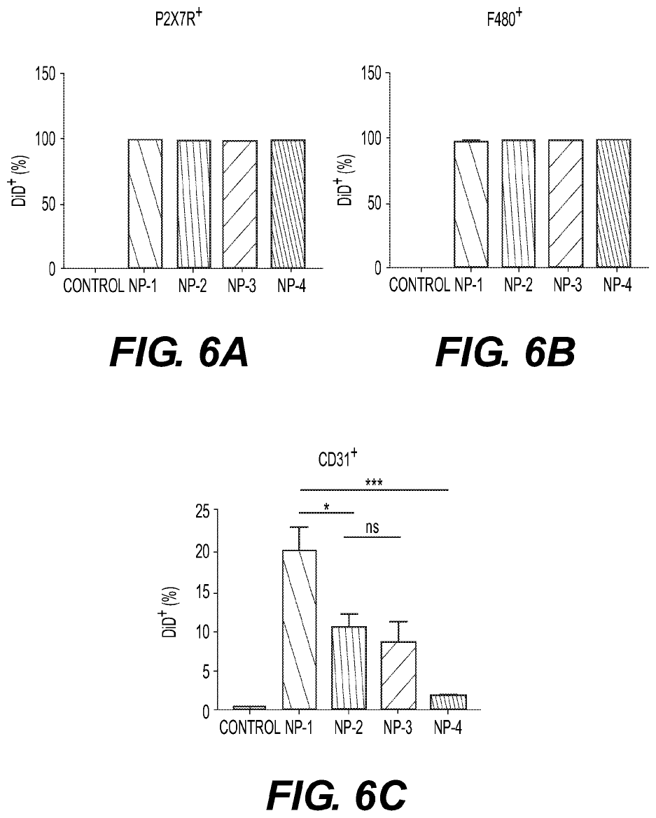

For all NP formulations, the percentage of DiD⁺ AEC I (FIG. 6A) and DiD⁺ alveolar macrophages (FIG. 6B) was significantly greater than the untreated control. The difference in the percentage of DiD⁺ endothelial cells between NP-1 and all other NP formulations were statistically significant, whereas the difference in the percentage of DiD⁺ endothelial cells between NP-2 and NP-3 were not statistically significant (FIG. 6C). All NP formulations had a significantly greater percentage of DiD⁺ endothelial cells than NP-4.

For all NP formulations, the percentage of DiD⁺ HSPCs (FIG. 8) was significantly greater than the untreated control. The difference in the percentage of DiD⁺ HSPCs between NP-1 and all other NP formulations were statistically significant, whereas the difference in the percentage of DiD⁺ HSPCs between NP-2, NP-3, and NP-4 were not statistically significant (FIG. 8).

(v) Flow Cytometry Exhibits Enhanced Uptake of NP-1 in Isolated Hematopoietic Stem and Progenitor Cells

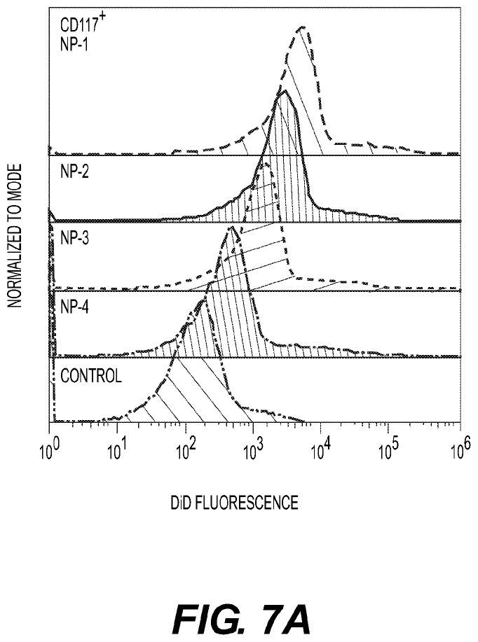

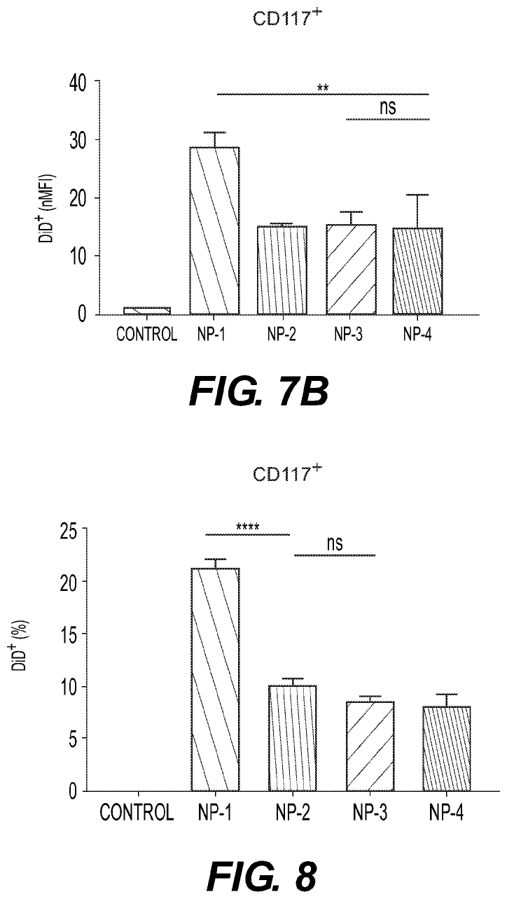

NP-1 demonstrated significant uptake in bulk bone marrow, so accumulation of NP-1 in HSPCs was investigated. Twenty-four h post-injection, bulk bone marrow was harvested and processed using the EasySep Mouse CD117 (cKIT) Positive Selection Kit. The majority (83.6%) of positively selected cells were confirmed as HPSCs (CD117⁺) by flow cytometry. NP-1, NP-2, NP-3, and NP-4 treated mice demonstrated an increase in DiD fluorescence in HSPCs compared to untreated controls (FIG. 7A). The nMFI in HPSC populations for NP-1 treated mice was significantly greater than NP-2, NP-3, and NP-4 treated mice (FIG. 7B). There was no significant difference in the nMFI of NP-2, NP-3, and NP-4 treated mice (FIG. 7B).

For all NP formulations, the percentage of DiD⁺ HSPCs (FIG. 8) was significantly greater than the untreated control. The difference in the percentage of DiD⁺ HSPCs between NP-1 and all other NP formulations were statistically significant, whereas the difference in the percentage of DiD⁺ HSPCs between NP-2, NP-3, and NP-4 were not statistically significant (FIG. 8).

The principal advantage of NPs as drug carriers is their small size, which allows them to traverse biological barriers, enter various tissues, and associate with specific cell populations. Size, therefore, is one of the main parameters that define the effectiveness of NPs for preferential delivery to desired cell populations. However, only a few studies of polymer NPs have carefully examined the effect of size on biodistribution (Cruz, et al., Journal of Controlled Release 2016, 223: 31-41; Yadav, et al., PDA J. Pharm. Sci. Technol. 2011, 65(2): 131-9; Kulkarni and Feng, Pharmaceutical Research 2013, 30(10): 2512-2522; He, et al., Biomaterials 2010, 31(13): 3657-3666; Vila, et al., International Journal of Pharmaceutics 2005, 292(1): 43-52; Liu, et al., Arch. Pharm. Res. 2008, 31(4): 547-554; Caster, et al., Nanomedicine: Nanotechnology, Biology and Medicine 2017, 13(5): 1673-1683). Rather, the influence of NP size on distribution in cells and tissues has been more thoroughly explored using inorganic NPs, given their ease of manufacturing in controlled size fractions (De Jong, et al., Biomaterials 2008, 29(12): 1912-1919; Zhang, et al., Biomaterials 2009, 30(10): 1928-1936). Size-dependent biodistribution was observed in a study of PEGylated gold NPs (Zhang, et al., Biomaterials 2009, 30(10): 1928-1936): small PEGylated gold NPs (20 nm) demonstrated significantly greater accumulation compared with 80 nm NPs in A341 tumor-xenografted mice. Outside of the tumor, 20 nm NPs had prolonged blood circulation and decreased uptake by the liver and spleen, while larger 80 nm NPs were taken up more readily by the liver and spleen (Zhang, et al., Biomaterials 2009, 30(10): 1928-1936).

It was surprising that results obtained with inorganic NPs are translatable to polymer NPs. PLGA NPs, for example, have been used to deliver a variety of therapeutic agents, including chemotherapy drugs, pDNA, siRNA, and PNAs (Sawyer, et al., Drug Delivery and Translational Research 2011, 1(1): 34-42; Malinovskaya, et al., International Journal of Pharmaceutics 2017, 524(1): 77-90; Bowerman, et al., Nano Letters 2017, 17(1): 242-248; Householder, et al., International Journal of Pharmaceutics 2015, 479(2): 374-380; Blum and Saltzman, Journal of Controlled Release 2008, 129(1): 66-72; Zhao, et al., PLOS ONE 2013. 8(12): e82648; Santos, et al., Nanotechnology, Biology and Medicine 2013, 9(7): 985-995; Woodrow, et al., Nature Materials 2009, 8: 526; Cun, et al., International Journal of Pharmaceutics 2010, 390(1): 70-75; McNeer, et al., Nature communications 2015, 6: 6952-6952; McNeer, et al., Molecular Therapy 2011, 19(1): 172-180; McNeer, et al., Gene Therapy 2012, 20: 658; Schleifman, et al., Molecular therapy. Nucleic Acids 2013, 2(11): e135-e135; Fields, et al., Advanced Healthcare Materials 2015, 4(3): 361-366; Bahal, et al., Nature Communications 2016, 7: 13304; Ricciardi, et al., Nature Communications 2018, 9(1): 2481). However, the effect of NP size (such as PLGA NP) on localization in tissues and cells after administration (such as injection) is still poorly understood. The paucity of information on such studies could be due to the difficulties in the scalable manufacturing of size-controlled NPs containing a biocompatible, biodegradable polymer, such as PLGA. The current study involves the synthesis of fluorescent, PLGA NPs of various sizes, using the NanoAssemblr™ microfluidic device. The NanoAssemblr™ allows for the control of several NP characteristics, including size by exploring operational parameters including flow rate ratio, flow rate, formulation volume, aqueous phase concentration, and organic phase concentration (Morikawa, et al., Biological and Pharmaceutical Bulletin 2018, 41(6): 899-907). Each parameter was varied to formulate reproducible NPs with a low PDI (FIGS. 1A-1C). It was discovered that decreasing the concentration of PLGA in the organic phase decreased the diameter of the NPs (FIG. 1C). Together, these data show that NP with specific average sizes can be synthesized using a scalable microfluidic approach.

It was hypothesized that intravenous injection of PLGA NPs of different sizes would result in altered in vivo biodistribution. To test this hypothesis, size-differentiated DiD-loaded NPs were injected retro-orbitally into mice. Twenty-four hours after injection, the accumulation of NPs in various tissues was assessed. This window of time was selected to ensure that a majority of the NPs were removed from circulation (Panagi, et al., International Journal of Pharmaceutics 2001, 221, 143-152). Two different methods were used to evaluate accumulation of NPs in organs: IVIS imaging and flow cytometry. While IVIS imaging allows for rapid assessment of biodistribution, this method is unable to resolve cellular uptake. Therefore, signals detected by IVIS may result from interstitial accumulation, rather than cell-specific uptake. Although flow cytometry requires additional processing steps prior to analysis, it was found that this technique more accurately predicts uptake by therapeutically relevant cell populations at the specified time point (Park, et al., Nanomedicine: Nanotechnology, Biology, and Medicine 2016, 12(5): 1365-1374; Cui, et al., Journal of Controlled Release 2019, 304, 259-267). Finally, confocal microscopy confirmed that NPs were internalized and accumulated in bone marrow and lung tissue.

Using the above the techniques, it was found that NP size greatly affected organ and cellular distribution. No significant accumulation at any NP size was observed in the brain and heart. In the pancreas and kidney, small NPs accumulated in the tissue to some extent, but few of those NPs were associated with cells strongly enough to observe them in flow cytometry (FIG. 4A). In the spleen and liver, the largest NPs have the highest levels of accumulation. In contrast, in the lung and bone marrow, the smallest NPs have the highest levels of accumulation, including accumulation within cell types that are of major interest in delivery of new therapies for lung and blood disorders.

For all NP formulations, the level of fluorescence detected in the heart and brain were insignificant. The low level of detection in the heart makes sense: biodistribution studies have demonstrated that polymer NPs >100 nm do not have significant uptake in heart tissue (Cruz, et al., Journal of Controlled Release 2016, 223: 31-41; Kulkarni and Feng, Pharmaceutical Research 2013, 30(10): 2512-2522). The low level of fluorescence observed in the brain may be explained via many earlier studies that have shown that passage from the systemic circulation to the brain through the blood-brain barrier (BBB) for unmodified PLGA NPs is low (<1%) (Li and Sabliov, Nanotechnology Reviews 2013, 2(3): 241-257; Lu, et al., Journal of Controlled Release 2007, 118(1): 38-53; Hu, et al., International Journal of Pharmaceutics 2011, 415(1): 273-283). Although there are reports of accumulation of certain NPs—particularly those that are decorated with certain surface ligands—into the brain, it is invariably a small fraction (˜1-2%) of the total injected dose (Saucier-Sawyer, et al., Journal of Drug Targeting 2015, 23(7-8): 736-749).

Using IVIS, the three smallest preparations (NP-1, NP-2, and NP-3) led to small, but significant, accumulation of fluorescence in the kidney. Using flow cytometry, however, only the smallest formulation (NP-1) resulted in a significant fluorescence signal. The discrepancy observed between these two methods is likely due to accumulation of NPs in the interstitial space, which would be detected by IVIS. However, preparation of tissues for flow cytometry requires a series of digestion, homogenization, and wash steps to create a single cell suspension. These additional processing steps would result in loss of any NPs residing in the interstitial space, while preserving NPs that have been taken up by cells.

Similarly, a discrepancy between IVIS and flow cytometry was observed, when analyzing the pancreas. By IVIS significant NP-1 accumulation was detected, but was not observed by flow cytometry. These results suggest that NP-1 particles were able to accumulate in the interstitium, but were not internalized by cells as measured by flow cytometry.

In contrast to these other tissues, both IVIS and flow cytometry revealed higher accumulation of the largest NPs in the spleen and liver. In the spleen, IVIS quantification and flow cytometry showed significant DiD fluorescence levels for the three largest NP formulations (NP-2, NP-3, and NP-4). These results align with previous studies that have shown that NPs with a diameter greater than 200 nm are rapidly removed from circulation and sequestered in the spleen (Albanese, et al., Annu. Rev. Biomed. Eng. 2012, 14(1): 1-16; Hoshyar, et al., Nanomedicine (London, England) 2016, 11(6): 673-692; Bertrand and Leroux, J Control Release 2012, 161(2): 152-63).

It is well known that NPs with a diameter greater than 200 nm are rapidly cleared from the blood stream by the liver (Albanese, et al., Annu. Rev. Biomed. Eng. 2012, 14(1): 1-16; Hoshyar, et al., Nanomedicine (London, England) 2016, 11(6): 673-692; Bertrand and Leroux, J Control Release 2012, 161(2): 15263). Further, inside the liver sinusoidal capillaries, Kupffer cells are responsible for the clearance of particulates, including NPs (Bertrand and Leroux, J Control Release 2012, 161(2): 15263). In previous work, using PLGA NPs that were similar in size to NP-3, it was shown that injected NPs are internalized by 98% of Kupffer cells, 89% of liver sinusoidal endothelial cells, 56% of hepatic stellate cells, and 7% of hepatocytes (Park, et al., Nanomedicine: Nanotechnology, Biology, and Medicine 2016, 12(5): 1365-1374). In the present study, it was demonstrated that the liver has the greatest level of NP-associated fluorescence by IVIS and flow cytometry. This high level of fluorescence is likely mediated by substantial NP uptake in Kupffer cells.

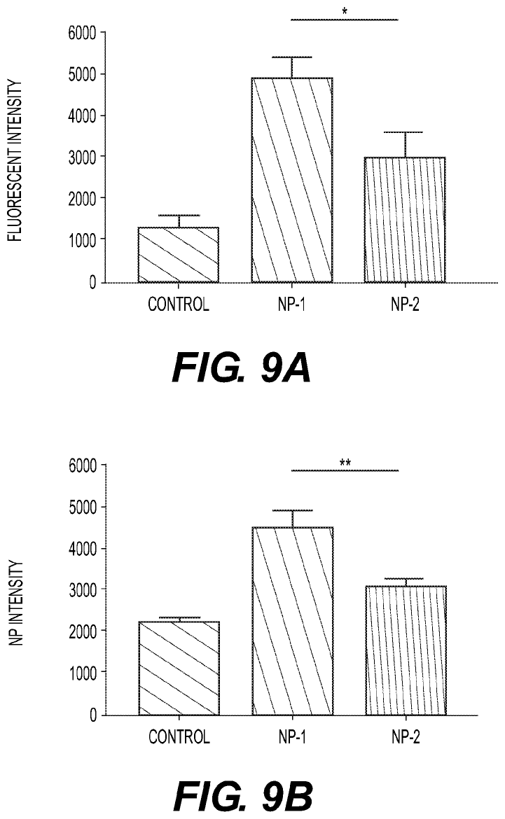

The smallest formulation, in terms of NP size, NP-1, demonstrated significantly enhanced uptake in the lung (FIG. 4C, FIG. 9A). Previous studies have shown that after intravenous injection, small NPs more readily accumulate in the lung when compared to larger NPs (Kulkarni and Feng, Pharmaceutical Research 2013, 30(10): 2512-2522). However, these prior studies looked at bulk biodistribution, using high performance liquid chromatography (HPLC) of tissue extracts, which are unable to assess biodistribution at a cellular level (Kulkarni and Feng, Pharmaceutical Research 2013, 30(10): 2512-2522). Therefore, it is unclear from this prior work whether NPs are gaining entry to parenchymal cells or simply accumulating in pulmonary interstitial or vasculature spaces. Previously, it was shown that flow cytometry is more sensitive than these extraction methods and could have the added benefit of distinguishing whether NP uptake is associated with particular cell populations (Fields, et al., Advanced Healthcare Materials 2015, 4(3): 361-366; Fields, et al., Journal of Controlled Release 2012, 164(1): 41-48). Here, flow cytometry was used to investigate whether NPs could associate with therapeutically relevant cell populations, including AEC I, alveolar macrophages, and endothelial cells. The smallest formulation, NP-1, demonstrated the highest level of DiD fluorescence by flow cytometry in AEC I and alveolar macrophages. These results show that NPs that are sufficiently small are able to successfully escape from the systemic circulation, through lung endothelial fenestrations, and into AEC I more readily than NPs with larger diameters. Interestingly, alveolar macrophages and epithelial cells have been shown to express the cystic fibrosis transmembrane conductance regulator (CFTR), a cAMP-dependent chloride channel, and may contribute to the hyperinflammatory immune response observed in patients diagnosed with cystic fibrosis (CF) (Di, et al., Nature Cell Biology 2006, 8: 933). The present study shows that therapeutic cargo can be effectively delivered to these cells in the lung using NPs approximately 120 nm in diameter, or perhaps even smaller.

Similar to the lung, the smallest NPs (NP-1) demonstrated significantly greater uptake in bone marrow compared with larger NPs (FIG. 9B). Further, flow cytometry was used to study whether NPs accessed HSPCs, a therapeutically pertinent cell population in the bone marrow. In HSPCs, NP-1 demonstrated the highest level of fluorescent uptake by flow cytometry, suggesting that small NPs escape from circulation, through bone marrow fenestrations, and into HSPCs more easily than larger NPs. Although the discontinuous endothelial fenestrations of the bone marrow have not been studied in detail, prior studies have shown that PLGA NPs with diameters of 150-300 nm accumulate in the bone marrow (McNeer, et al., Gene Therapy 2012, 20: 658; Swami, et al., Proceedings of the National Academy of Sciences 2014, 111(28): 10287). Also, previous work used PLGA NPs approximately 300 nm in diameter to deliver PNA/donor DNA combinations to the bone marrow to edit HSPCs in a mouse model of β-thalassemia (Bahal, et al., Nature Communications 2016, 7: 13304; Ricciardi, et al., Nature Communications 2018, 9(1): 2481). While successful gene modification using these particles (similar in size to NP-3) has been demonstrated, it is not known whether a formulation of containing a high percentage of smaller size NPs, such as a size similar to that of NP-1, could show improved NP uptake by cells and/or delivery of PNA/donor DNA to bone marrow.

The present results show that IVIS quantification and flow cytometry do not always provide the same information. Here, IVIS served as a preliminary screening method to detect NP fluorescence in intact organs, while flow cytometry was used to measure fluorescence associated with individual cells. While useful as a general screening tool, IVIS imaging does not differentiate among NPs remaining in the vasculature, distributed in the interstitial space, or associated with cells. Flow cytometry, on the other hand, accurately provides information on cellular distribution, but limits understanding of broad NP distribution in whole organs or associated vasculature. Biodistribution 24 hr after administration (such as IV injection) was investigated. It is known from prior studies that PLGA NPs are cleared from circulation by this time (Panagi, et al., International Journal of Pharmaceutics 2001, 221(1): 143-152.). Therefore, when paired together with confocal microscopy, these complementary methods can provide a comprehensive understanding of NP biodistribution, and their localization in cells or interstitial spaces.

(vi) Release Kinetics of DiD

The release kinetics of DiD from the nanoparticles was investigated to determine whether the IVIS method was actually tracking nanoparticles and not DiD released from the nanoparticles, using NP-1 and NP-2. NP-1 and NP-2 demonstrated DiD loading of 9 μg per mg of NPs, with less than 2% release over 24 h. This negligible release shows that the IVIS method tracked the nanoparticles and, therefore, confirmed that the biodistribution data show the distribution of the nanoparticles and not an agent released from the nanoparticles.

(vii) Confocal Imaging Shows Cellular Internalization of Nanoparticles

To further confirm that cells internalize NPs, confocal microscopy was performed in bone marrow and lung tissue. Twenty-four hrs after injection, bulk bone marrow cells were harvested, fixed, and stained with Hoechst 33342 for imaging and quantification. Visually, NPs overlapped with the blue Hoechst staining of the cellular DNA. Further, NP-1 demonstrated enhanced internalization and accumulation within bone marrow cells after administration when compared to NP-2. When quantified, NP-1 had an average fluorescent intensity of ˜5000, a nearly two-fold (and statistically significant) increase over NP-2 (FIG. 9A). To visualize NP internalization in lung tissue, tissues were harvested, frozen in O.C.T., and sectioned 24 h after injection. NP-1 demonstrated greater internalization and accumulation within lung cells after administration when compared to NP-2. When this was quantified, NP-1 had an average NP intensity of ˜4500, nearly two-fold greater than NP-2, which was significantly different (FIG. 9B).

### Example 2: In Vivo Gene Editing

### Materials and Methods

For in vivo gene editing experiments, transgenic mice containing the IVS2-654 β-thalassemic mutation were employed (Svasti, et al., Proc. Natl. Acad. Sci. USA 2009, 106(4), 1205-1210; Bahal, et al., Nat. Commun. 2016, 7, 13304). Three hours prior to each nanoparticle injection, 12 ug (480 ug kg⁻¹) of stem cell factor (SCF) was injected intra-peritoneally. Following administration of SCF, each mouse received 2 mg of nanoparticles via retro-orbital injection. In total, 4 doses of SCF and nanoparticles were administered in 48 hour intervals. Two weeks after the final dose of SCF and nanoparticles were administered, mice were scarified and tissues were harvested. Where indicated, hematopoietic stem and progenitor cells (HSPCs) were enriched via lineage depletion using the EasySep Mouse Hematopoietic Progenitor Cell Isolation Kit (STEMCELL). Epithelium from the lungs and livers were enriched using the EasySep Mouse Epithelial Cell Enrichment Kit II (STEMCELL). In all cases, genomic DNA was harvested from single cell suspensions using the ReliaPrep gDNA Tissue Miniprep System (Promega) and analyzed for editing frequencies via digital droplet PCR (BioRad).

### Results

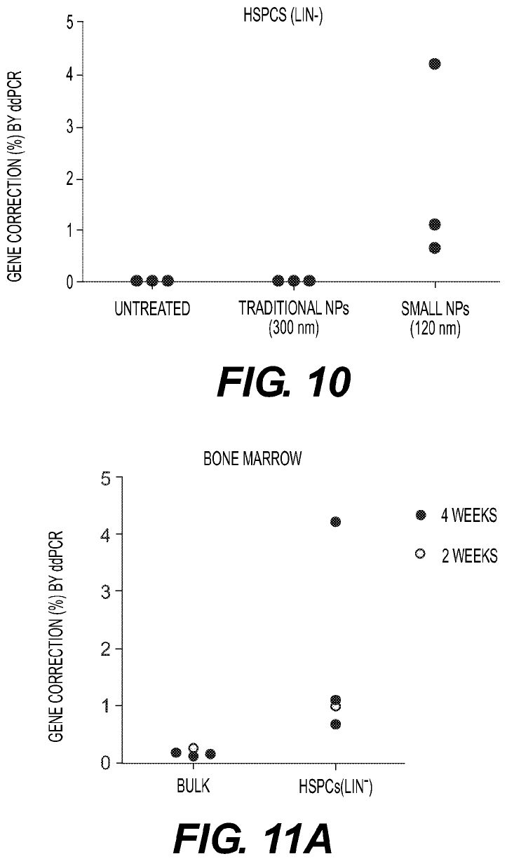

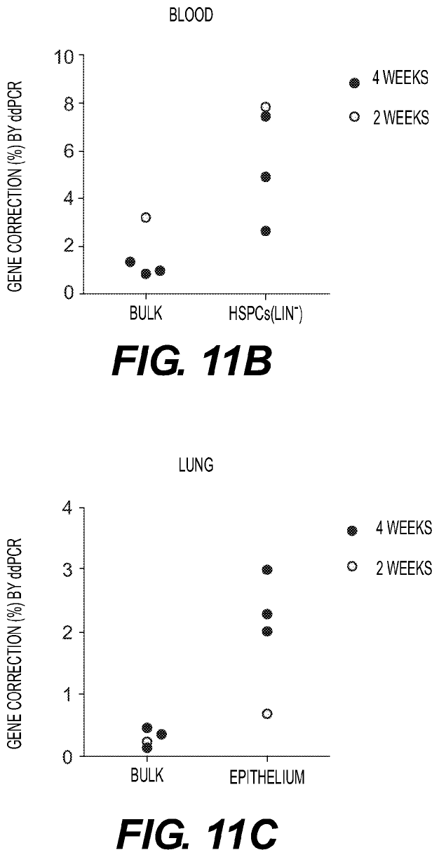

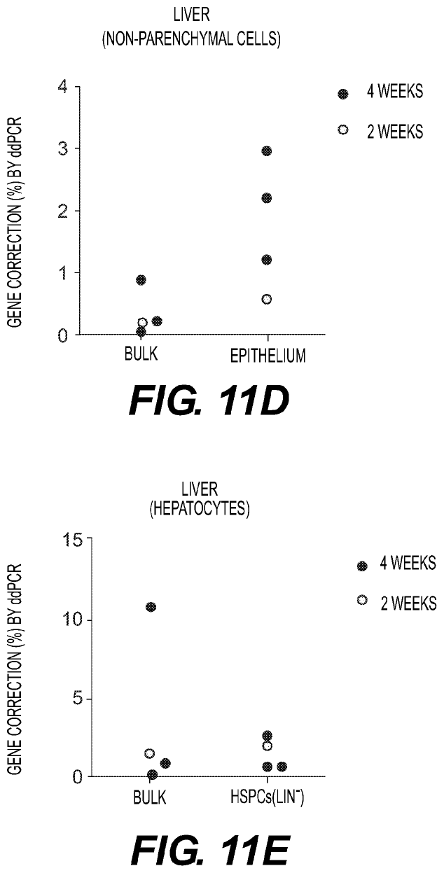

The results are shown in FIG. 10, and FIGS. 11A-11E.

The NanoAssemblr™, a scalable microfluidic platform, can be used to engineer size-differentiated biocompatible NPs. After intravenous injection, small (˜120 nm) NPs demonstrate significantly greater uptake in the lung and bone marrow compared with larger NPs. Further, these small, 120 nm NPs associated with AEC I and alveolar macrophages in the lung, and HPSCs in the bone marrow more readily than larger NPs. Thus, a population of NPs of having a high proportion of NPs with size similar to NP-1 is more effective for improved delivery of agents to therapeutically pertinent tissues, by avoiding sequestration in the liver and spleen, and by crossing tissue barriers to reach relevant cellular targets. This study demonstrates using biodegradable NP size to passively target tissues and specific sub-cellular populations.

1. A population of nanoparticles having a diameter between about 50 nm and about 350 nm, wherein at least 85% of the nanoparticles have a diameter between about 120 nm and about 145 nm;
wherein the nanoparticles comprise biocompatible biodegradable polymers; and wherein a subset or all of the nanoparticles comprise therapeutic agents, diagnostic agents, and/or prophylactic agents.

2. The population of nanoparticles of claim 1, wherein the nanoparticles are selectively taken up by lung cells and/or bone marrow cells of a mammal, as measured by flow cytometry.

3. The population of nanoparticles of claim 2, wherein the lung cells include or are type I alveolar epithelial cells and/or alveolar macrophage cells.

4. The population of nanoparticles of claim 2, wherein the bone marrow cells include or are hematopoietic stem and progenitor cells.

5. The population of nanoparticles of claim 1, wherein the population of nanoparticles have a diameter between about 70 nm and about 300 nm, preferably between about 70 nm and about 220 nm.

6. The population of nanoparticles of claim 1, wherein at least 90% of the nanoparticles have a diameter between about 120 nm and about 145 nm, preferably between about 125 nm and about 140 nm.

7. The population of nanoparticles of claim 1, wherein at least 90% of the nanoparticles have a diameter between about 100 nm and about 135 nm, preferably between about 110 nm and about 129 nm.

8. The population of nanoparticles of claim 1, wherein the nanoparticles have a polydispersity index less than 0.25.

9. The population of nanoparticles of claim 1, wherein the biodegradable polymers comprise a hydrophobic polymer; a hydrophilic polymer; an amphiphilic polymer comprising a hydrophobic polymer portion and a hydrophilic polymer portion; co-polymers; or blends thereof.

10. The population of nanoparticles of claim 9, wherein the hydrophobic polymer or hydrophobic polymer portion comprises a polyester, poly(anhydride), poly(orthoester), hydrophobic polypeptide, polyamide, poly(ester-amide), poly(beta-amino ester)s, poly(amine-co-ester)s; poly(amine-co-ester-co-ortho ester)s, poly(alkyl acrylate) (such as poly (methyl acrylate)); poly(alkyl alkacrylate) (such as poly (methyl methacrylate)); poly(alkyl acrylamide) (such as poly (N-isopropyl acrylamide)); poly(alkyl alkacrylamide) (such as poly (N-isopropyl methacrylamide)), alkyl cellulose, cellulose ester, polyurethane, polyurea, poly(urea ester), poly(amide-enamine), hydrophobic polyethers (such as polypropylene glycol), or copolymers thereof.

11. The population of nanoparticles of claim 9, wherein the hydrophobic polymer or hydrophobic polymer portion comprises a polyester, preferably a hydrophobic poly(hydroxy acid).

12. The population of nanoparticles of claim 9, wherein the hydrophobic polymer or hydrophobic polymer portion comprises poly(lactic acid-co-glycolic acid), poly(lactic acid), or poly(glycolic acid).

13. The population of nanoparticles of claim 9, wherein the hydrophilic polymer or hydrophilic polymer portion comprises polyalkylene glycol such as polyethylene glycol (PEG); polysaccharides such as cellulose and starch; hydrophilic polypeptides such as poly-L-glutamic acid, gamma-polyglutamic acid, poly-L-aspartic acid, poly-L-serine, or poly-L-lysine; poly(oxyethylated polyol); poly(olefinic alcohol) such as poly(vinyl alcohol); poly(vinylpyrrolidone); poly(N-hydroxyalkyl methacrylamide) such as poly(N-hydroxyethyl methacrylamide); poly(N-hydroxyalkyl methacrylate) such as poly(N-hydroxyethyl methacrylate); hydrophilic poly(hydroxy acids); and copolymers thereof.

14. The population of nanoparticles of claim 1, wherein the nanoparticles containing therapeutic agents, diagnostic agents, prophylactic agents in a loading between about 0.2 mg/mL and about 5 mg/mL, between about 0.2 mg/mL and about 2 mg/mL, between about 0.2 mg/mL and about 1 mg/mL, as measured by absorbance.

15. The population of nanoparticles of claim 1, wherein therapeutic agents, diagnostic agents, prophylactic agents comprise a nucleic acid, protein, peptide, lipid, polysaccharide, small molecules, or combination thereof.

16. The population of nanoparticles of claim 1, wherein therapeutic agents, diagnostic agents, prophylactic agents comprise nucleic acid, preferably selected from the group consisting of a peptide nucleic acid (PNA), deoxyribonucleic acid (DNA), preferably a donor DNA, ribonucleic acid (RNA), and combinations thereof.

17. The population of nanoparticles of claim 1, wherein the therapeutic, diagnostic, and/or prophylactic agent comprises a combination PNA and donor DNA.

18. The population of nanoparticles of claim 16 or 17, wherein the PNA, DNA, preferably donor DNA, and/or RNA are oligonucleotides.

19. The population of nanoparticles of claim 1, wherein some or all of the nanoparticles do not contain a targeting agent on their surface.

20. A pharmaceutical composition comprising the population of nanoparticles of claim 1 and a pharmaceutically acceptable carrier.

21. A method of treating a subject in need thereof comprising administering to the subject an effective amount of the composition of claim 20.

22. The method of claim 21, wherein the composition is formulated for parenteral administration, preferably intravenous delivery.

23. The method of claim 21, wherein the subject has a lung disorder or blood disorder.

24. A method of making the population of nanoparticles of claim 1 using a microfluidic system, the method comprising:
(i) providing a first fluid comprising the biodegradable polymer into a first channel of the microfluidic system; or
(ii) providing a second fluid comprising a non-solvent of the biodegradable polymer into a second channel of the microfluidic system;

25. The method of claim 24, wherein (i) and (ii) are performed simultaneously, or in any order, and wherein the first and second fluids contact downstream to form the population of nanoparticles.

26. The method of claim 24 or 25, wherein the first fluid comprises the therapeutic agents, diagnostic agents, prophylactic agents.

27. The method of claim 24, wherein the first fluid comprises an organic solvent or solution.

28. The method of claim 24, wherein the non-solvent of the biodegradable polymer is an aqueous solution.

29. The method of claim 24, wherein the second fluid comprises a surfactant, preferably poly(vinyl alcohol).

30. The method of claim 24, wherein the first fluid and second fluid have a flow rate ratio between 1:10 and 10:1, inclusive.

31. The method of claim 24, wherein the first fluid and the second fluid independently have flow rates between 1 mL/min and 20 mL/min, inclusive.

32. The method of claim 24, wherein the first fluid and the second fluid independently have formulation volumes between 1 mL and 10 mL, inclusive.

33. The method of claim 24, wherein the first fluid has a concentration between 1 mg/mL and 250 mg/mL.

34. The method of claim 24, wherein the second fluid has a concentration between 0.1% w/v and 5% w/v, inclusive.


SEQUENCE LISTING (116 sequences)

SEQ ID NO: 1 - DNA, 12 bp, Artificial Sequence
  Synthetic Primer
gaaagaaagaga

SEQ ID NO: 2 - DNA, 18 bp, Artificial Sequence
  Synthetic Primer
tgccctgaaagaaagaga

SEQ ID NO: 3 - DNA, 7 bp, Artificial Sequence
  Synthetic Primer
ggagaaa

SEQ ID NO: 4 - DNA, 17 bp, Artificial Sequence
  Synthetic Primer
agaatggtgcaaagagg

SEQ ID NO: 5 - DNA, 7 bp, Artificial Sequence
  Synthetic Primer
aaaaggg

SEQ ID NO: 6 - DNA, 18 bp, Artificial Sequence
  Synthetic Primer
acatgattagcaaaaggg

SEQ ID NO: 7 - DNA, 12 bp, Artificial Sequence
  Synthetic Primer
ctttctttctct

SEQ ID NO: 8 - DNA, 12 bp, Artificial Sequence
  Synthetic Primer
tctctttctttc

SEQ ID NO: 9 - DNA, 18 bp, Artificial Sequence
  Synthetic Primer
tctctttctttcagggca

SEQ ID NO: 10 - DNA, 6 bp, Artificial Sequence
  Synthetic Primer
tttccc

SEQ ID NO: 11 - DNA, 7 bp, Artificial Sequence
  Synthetic Primer
ccctttt

SEQ ID NO: 12 - DNA, 18 bp, Artificial Sequence
  Synthetic Primer
cccttttgctaatcatgt

SEQ ID NO: 13 - DNA, 7 bp, Artificial Sequence
  Synthetic Primer
tttctcc

SEQ ID NO: 14 - DNA, 7 bp, Artificial Sequence
  Synthetic Primer
cctcttt

SEQ ID NO: 15 - DNA, 17 bp, Artificial Sequence
  Synthetic Primer
cctctttgcaccattct

SEQ ID NO: 16 - DNA, 12 bp, Artificial Sequence
  Synthetic Primer
  misc_feature (1)..(1): n = Pseudoisocytosine
  misc_feature (5)..(5): n = Pseudoisocytosine
  misc_feature (9)..(9): n = Pseudoisocytosine
  misc_feature (11)..(11): n = Pseudoisocytosine
ntttntttntnt

SEQ ID NO: 17 - DNA, 7 bp, Artificial Sequence
  Synthetic Primer
  misc_feature (5)..(7): n = Pseudoisocytosine
ttttnnn

SEQ ID NO: 18 - DNA, 7 bp, Artificial Sequence
  Synthetic Primer
  misc_feature (4)..(4): n = Pseudoisocytosine
  misc_feature (6)..(7): n = Pseudoisocytosine
tttntnn

SEQ ID NO: 19 - DNA, 30 bp, Artificial Sequence
  Synthetic Peptide Nucleic Acid
  misc_feature (1)..(1): n = Pseudoisocytosine
  misc_feature (1)..(1): Linked to lys-lys-lys
  misc_feature (5)..(5): n = Pseudoisocytosine
  misc_feature (9)..(9): n = Pseudoisocytosine
  misc_feature (11)..(11): n = Pseudoisocytosine
  misc_feature (12)..(13): Linked by three 8-amino-2, 6, 10-trioxaoctanoic acid, three 8-amino-3,6-dioxaoctanoic acid, or three 6-aminohexanoic acid molecules
  misc_feature (30)..(30): Linked to lys-lys-lys
ntttntttntnttctctttctttcagggca

SEQ ID NO: 20 - DNA, 25 bp, Artificial Sequence
  Synthetic Peptide Nucleic Acid
  misc_feature (1)..(1): Linked to lys-lys-lys
  misc_feature (5)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ttttnnncccttttgctaatcatgt

SEQ ID NO: 21 - DNA, 24 bp, Artificial Sequence
  Synthetic Peptide Nucleic Acid
  misc_feature (1)..(1): Linked to lys-lys-lys
  misc_feature (4)..(4): n = Pseudoisocytosine
  misc_feature (6)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (24)..(24): Linked to lys-lys-lys
tttntnncctctttgcaccattct

SEQ ID NO: 22 - DNA, 10 bp, Artificial Sequence
  Synthetic Primer
  misc_feature (2)..(2): n = Pseudoisocytosine
  misc_feature (7)..(7): n = Pseudoisocytosine
  misc_feature (10)..(10): n = Pseudoisocytosine
tnttttnttn

SEQ ID NO: 23 - DNA, 10 bp, Artificial Sequence
  Synthetic Primer
cttcttttct

SEQ ID NO: 24 - DNA, 10 bp, Artificial Sequence
  Synthetic Primer
  misc_feature (3)..(3): n = Pseudoisocytosine
  misc_feature (6)..(6): n = Pseudoisocytosine
  misc_feature (10)..(10): n = Pseudoisocytosine
ttnttntttn

SEQ ID NO: 25 - DNA, 10 bp, Artificial Sequence
  Synthetic Primer
ctttcttctt

SEQ ID NO: 26 - DNA, 10 bp, Artificial Sequence
  Synthetic Primer
  misc_feature (1)..(3): n = Pseudoisocytosine
  misc_feature (5)..(6): n = Pseudoisocytosine
  misc_feature (9)..(9): n = Pseudoisocytosine
nnntnnttnt

SEQ ID NO: 27 - DNA, 10 bp, Artificial Sequence
  Synthetic Primer
tcttcctccc

SEQ ID NO: 28 - DNA, 20 bp, Artificial Sequence
  Synthetic Peptide Nucleic Acid
  misc_feature (1)..(1): Linked to lys-lys-lys
  misc_feature (3)..(3): n = Pseudoisocytosine
  misc_feature (6)..(6): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
ttnttntttnctttcttctt

SEQ ID NO: 29 - DNA, 20 bp, Artificial Sequence
  Synthetic Peptide Nucleic Acid
  misc_feature (1)..(1): Linked to lys-lys-lys
  misc_feature (1)..(3): n = Pseudoisocytosine
  misc_feature (5)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
nnntnnttnttcttcctccc

SEQ ID NO: 30 - DNA, 7 bp, Artificial Sequence
  Synthetic Primer
cctcttc

SEQ ID NO: 31 - DNA, 7 bp, Artificial Sequence
  Synthetic Primer
cttctcc

SEQ ID NO: 32 - DNA, 15 bp, Artificial Sequence
  Synthetic Primer
cttctccaaaggagt

SEQ ID NO: 33 - DNA, 18 bp, Artificial Sequence
  Synthetic Primer
cttctccacaggagtcag

SEQ ID NO: 34 - DNA, 22 bp, Artificial Sequence
  synthetic polynucleotide
cttctccacaggagtcaggtgc

SEQ ID NO: 35 - DNA, 7 bp, Artificial Sequence
  Synthetic Primer
ttcctct

SEQ ID NO: 36 - DNA, 7 bp, Artificial Sequence
  Synthetic Primer
tctcctt

SEQ ID NO: 37 - DNA, 15 bp, Artificial Sequence
  Synthetic Primer
tctccttaaacctgt

SEQ ID NO: 38 - DNA, 18 bp, Artificial Sequence
  synthetic polynucleotide
tctccttaaacctgtctt

SEQ ID NO: 39 - DNA, 8 bp, Artificial Sequence
  Synthetic Primer
tctcttct

SEQ ID NO: 40 - DNA, 8 bp, Artificial Sequence
  Synthetic Primer
tcttctct

SEQ ID NO: 41 - DNA, 16 bp, Artificial Sequence
  Synthetic Primer
tcttctctgtctccac

SEQ ID NO: 42 - DNA, 20 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (7)..(7): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
tnttttnttncttcttttct

SEQ ID NO: 43 - DNA, 7 bp, Artificial Sequence
  Synthetic Primer
  misc_feature (1)..(2): n = Pseudoisocytosine
  misc_feature (4)..(4): n = Pseudoisocytosine
  misc_feature (7)..(7): n = Pseudoisocytosine
nntnttn

SEQ ID NO: 44 - DNA, 22 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
nntnttncttctccaaaggagt

SEQ ID NO: 45 - DNA, 22 bp, Artificial Sequence
  Synthetic Peptide Nucleic Acid
  misc_feature (1)..(1): Linked to lys-lys-lys
  misc_feature (3)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
ttnntnttctccttaaacctgt

SEQ ID NO: 46 - DNA, 25 bp, Artificial Sequence
  Synthetic Peptide Nucleic Acid
  misc_feature (1)..(1): Linked to lys-lys-lys
  misc_feature (3)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ttnntnttctccttaaacctgtctt

SEQ ID NO: 47 - DNA, 24 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (24)..(24): Linked to lys-lys-lys
tntnttnttcttctctgtctccac

SEQ ID NO: 48 - DNA, 25 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
nntnttncttctccacaggagtcag

SEQ ID NO: 49 - DNA, 29 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (29)..(29): Linked to lys-lys-lys
nntnttncttctccacaggagtcaggtgc

SEQ ID NO: 50 - DNA, 17 bp, Artificial Sequence
  Synthetic Primer
cttacccatagaggaaa

SEQ ID NO: 51 - DNA, 60 bp, Artificial Sequence
  Synthetic Primer
aaagaataacagtgataatttctgggttaaggcaatagcaatatctctgcatataaatat

SEQ ID NO: 52 - DNA, 51 bp, Artificial Sequence
  Synthetic Primer
gttcagcgtgtccggcgagggcgaggtgagtctatgggacccttgatgttt

SEQ ID NO: 53 - DNA, 51 bp, Artificial Sequence
  Synthetic Primer
aaacatcaagggtcccatagactcacctcgccctcgccggacacgctgaac

SEQ ID NO: 54 - DNA, 70 bp, Artificial Sequence
  Synthetic Primer
cttgccccacagggcagtaacggcagatttttcttccggcgttaaatgcaccatggtgtctgtttgaggt

SEQ ID NO: 55 - DNA, 51 bp, Artificial Sequence
  Synthetic Primer
acagacaccatggtgcacctgactcctgaggagaagtctgccgttactgcc

SEQ ID NO: 56 - DNA, 65 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (62)..(63): Optional phosphorothioate internucleoside linkage
  misc_feature (63)..(64): Optional phosphorothioate internucleoside linkage
  misc_feature (64)..(65): Optional phosphorothioate internucleoside linkage
ttgccccacagggcagtaacggcagacttctcctcaggagtcaggtgcaccatggtgtctgtttg

SEQ ID NO: 57 - DNA, 8 bp, Artificial Sequence
  Synthetic Primer
tttcctct

SEQ ID NO: 58 - DNA, 17 bp, Artificial Sequence
  Synthetic Primer
tttcctctatgggtaag

SEQ ID NO: 59 - DNA, 8 bp, Artificial Sequence
  Synthetic Primer
agaggaaa

SEQ ID NO: 60 - DNA, 26 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (26)..(26): Linked to lys-lys-lys
tntnttnttcttctctgtctccacat

SEQ ID NO: 61 - DNA, 8 bp, Artificial Sequence
  Synthetic Primer
agaagagg

SEQ ID NO: 62 - DNA, 17 bp, Artificial Sequence
  Synthetic Primer
atgccaactagaagagg

SEQ ID NO: 63 - DNA, 8 bp, Artificial Sequence
  Synthetic Primer
cctcttct

SEQ ID NO: 64 - DNA, 17 bp, Artificial Sequence
  Synthetic Primer
cctcttctagttggcat

SEQ ID NO: 65 - DNA, 9 bp, Artificial Sequence
  Synthetic Primer
ctttccctt

SEQ ID NO: 66 - DNA, 18 bp, Artificial Sequence
  Synthetic Primer
ctttcccttgtatctttt

SEQ ID NO: 67 - DNA, 9 bp, Artificial Sequence
  Synthetic Primer
aagggaaag

SEQ ID NO: 68 - DNA, 18 bp, Artificial Sequence
  Synthetic Primer
aaaagatacaagggaaag

SEQ ID NO: 69 - DNA, 10 bp, Artificial Sequence
  Synthetic Primer
gaaggagaaa

SEQ ID NO: 70 - DNA, 7 bp, Artificial Sequence
  Synthetic Primer
aaaagga

SEQ ID NO: 71 - DNA, 10 bp, Artificial Sequence
  Synthetic Primer
agaaaaaagg

SEQ ID NO: 72 - DNA, 7 bp, Artificial Sequence
  Synthetic Primer
agaaaaa

SEQ ID NO: 73 - DNA, 9 bp, Artificial Sequence
  Synthetic Primer
agagaaaga

SEQ ID NO: 74 - DNA, 7 bp, Artificial Sequence
  Synthetic Primer
aaagaaa

SEQ ID NO: 75 - DNA, 8 bp, Artificial Sequence
  Synthetic Primer
tctccttt

SEQ ID NO: 76 - DNA, 9 bp, Artificial Sequence
  Synthetic Primer
ttccctttc

SEQ ID NO: 77 - DNA, 8 bp, Artificial Sequence
  Synthetic Primer
  misc_feature (2)..(2): n = Pseudoisocytosine
  misc_feature (4)..(5): n = Pseudoisocytosine
tntnnttt

SEQ ID NO: 78 - DNA, 8 bp, Artificial Sequence
  Synthetic Primer
tcttctcc

SEQ ID NO: 79 - DNA, 18 bp, Artificial Sequence
  Synthetic Primer
tcttctctgtctccacat

SEQ ID NO: 80 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tnttttttttttctgtaatttttaa

SEQ ID NO: 81 - DNA, 26 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (8)..(8): n = Pseudoisocytosine
  misc_feature (9)..(10): Linked by three 8-amino-2, 6, 10-trioxaoctanoic acid, three 8-amino-3,6-dioxaoctanoic acid, or three 6-aminohexanoic acid molecules
  misc_feature (26)..(26): Linked to lys-lys-lys
tntntttnttctttctctgcaaactt

SEQ ID NO: 82 - DNA, 25 bp, Artificial Sequence
  Synthetic Peptide Nucleic Acid
  misc_feature (1)..(1): Linked to lys-lys-lys
  misc_feature (4)..(4): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tttnttttttctttaagaacgagca

SEQ ID NO: 83 - DNA, 60 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (57)..(58): Optional phosphorothioate internucleoside linkage
  misc_feature (58)..(59): Optional phosphorothioate internucleoside linkage
  misc_feature (59)..(60): Optional phosphorothioate internucleoside linkage
aagtttgcagagaaagataatatagtccttggagaaggaggaatcaccctgagtggaggt

SEQ ID NO: 84 - DNA, 8 bp, Artificial Sequence
  synthetic polynucleotide
  misc_feature (2)..(2): n = Pseudoisocytosine
  misc_feature (5)..(5): n = Pseudoisocytosine
  misc_feature (7)..(8): n = Pseudoisocytosine
tnttntnn

SEQ ID NO: 85 - DNA, 9 bp, Artificial Sequence
  Synthetic Primer
  misc_feature (3)..(5): n = Pseudoisocytosine
  misc_feature (9)..(9): n = Pseudoisocytosine
ttnnntttn

SEQ ID NO: 86 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(5): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tntnnttttttcctctatgggtaag

SEQ ID NO: 87 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (5)..(5): n = Pseudoisocytosine
  misc_feature (7)..(8): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tnttntnncctcttctagttggcat

SEQ ID NO: 88 - DNA, 27 bp, Artificial Sequence
  Synthetic Peptide Nucleic Acid
  misc_feature (1)..(1): Linked to lys-lys-lys
  misc_feature (3)..(5): n = Pseudoisocytosine
  misc_feature (9)..(9): n = Pseudoisocytosine
  misc_feature (9)..(10): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
ttnnntttnctttcccttgtatctttt

SEQ ID NO: 89 - DNA, 61 bp, Artificial Sequence
  Synthetic Primer
ttctgtatctatattcatcataggaaacaccaaagataatgttctccttaatggtgccagg

SEQ ID NO: 90 - DNA, 10 bp, Artificial Sequence
  Synthetic Primer
cttcctcttt

SEQ ID NO: 91 - DNA, 10 bp, Artificial Sequence
  Synthetic Primer
tttctccttc

SEQ ID NO: 92 - DNA, 18 bp, Artificial Sequence
  Synthetic Primer
tttctccttcagtgttca

SEQ ID NO: 93 - DNA, 7 bp, Artificial Sequence
  Synthetic Primer
ttttcct

SEQ ID NO: 94 - DNA, 7 bp, Artificial Sequence
  Synthetic Primer
tcctttt

SEQ ID NO: 95 - DNA, 20 bp, Artificial Sequence
  Synthetic Primer
tccttttgctcacctgtggt

SEQ ID NO: 96 - DNA, 10 bp, Artificial Sequence
  Synthetic Primer
tcttttttcc

SEQ ID NO: 97 - DNA, 10 bp, Artificial Sequence
  Synthetic Primer
ccttttttct

SEQ ID NO: 98 - DNA, 18 bp, Artificial Sequence
  Synthetic Primer
ccttttttctggctaagt

SEQ ID NO: 99 - DNA, 10 bp, Artificial Sequence
  Synthetic Primer
  misc_feature (1)..(1): n = Pseudoisocytosine
  misc_feature (4)..(5): n = Pseudoisocytosine
  misc_feature (7)..(7): n = Pseudoisocytosine
nttnntnttt

SEQ ID NO: 100 - DNA, 7 bp, Artificial Sequence
  Synthetic Primer
  misc_feature (5)..(6): n = Pseudoisocytosine
ttttnnt

SEQ ID NO: 101 - DNA, 10 bp, Artificial Sequence
  Synthetic Primer
  misc_feature (2)..(2): n = Pseudoisocytosine
  misc_feature (9)..(10): n = Pseudoisocytosine
tnttttttnn

SEQ ID NO: 102 - DNA, 28 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (4)..(5): n = Pseudoisocytosine
  misc_feature (7)..(7): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (29)..(29): Linked to lys-lys-lys
nttnntnttttttctccttcagtgttca

SEQ ID NO: 103 - DNA, 27 bp, Artificial Sequence
  Synthetic Peptide Nucleic Acid
  misc_feature (1)..(1): Linked to lys-lys-lys
  misc_feature (5)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
ttttnnttccttttgctcacctgtggt

SEQ ID NO: 104 - DNA, 28 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (9)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (28)..(28): Linked to lys-lys-lys
tnttttttnnccttttttctggctaagt

SEQ ID NO: 105 - DNA, 58 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (55)..(56): Optional phosphorothioate internucleoside linkage
  misc_feature (56)..(57): Optional phosphorothioate internucleoside linkage
  misc_feature (57)..(58): Optional phosphorothioate internucleoside linkage
tgggattcaataaccttgcagacagtggaggaaggcctttggcgtgataccacaggtg

SEQ ID NO: 106 - DNA, 7 bp, Artificial Sequence
  Synthetic Primer
tcttttt

SEQ ID NO: 107 - DNA, 7 bp, Artificial Sequence
  Synthetic Primer
tttttct

SEQ ID NO: 108 - DNA, 18 bp, Artificial Sequence
  Synthetic Primer
tttttctgtaatttttaa

SEQ ID NO: 109 - DNA, 9 bp, Artificial Sequence
  Synthetic Primer
tctctttct

SEQ ID NO: 110 - DNA, 9 bp, Artificial Sequence
  Synthetic Primer
tctttctct

SEQ ID NO: 111 - DNA, 17 bp, Artificial Sequence
  Synthetic Primer
tctttctctgcaaactt

SEQ ID NO: 112 - DNA, 7 bp, Artificial Sequence
  Synthetic Primer
tttcttt

SEQ ID NO: 113 - DNA, 18 bp, Artificial Sequence
  Synthetic Primer
tttctttaagaacgagca

SEQ ID NO: 114 - DNA, 7 bp, Artificial Sequence
  Synthetic Primer
  misc_feature (2)..(2): n = Pseudoisocytosine
tnttttt

SEQ ID NO: 115 - DNA, 9 bp, Artificial Sequence
  Synthetic Primer
  misc_feature (2)..(2): n = Pseudoisocytosine
  misc_feature (4)..(4): n = Pseudoisocytosine
  misc_feature (8)..(8): n = Pseudoisocytosine
tntntttnt

SEQ ID NO: 116 - DNA, 7 bp, Artificial Sequence
  Synthetic Primer
  misc_feature (4)..(4): n = Pseudoisocytosine
tttnttt